(19) United States
(12) Patent Application Publication
(10) Pub. No.: US 2008/0075734 A1
(43) Pub. Date: Mar. 27, 2008

(54) Genetic Immunization

(76) Inventors: Zane C. Neal, Cambria, WI (US); Hans Herweijer, Brooklyn, WI (US)

Correspondence Address: MIRUS CORPORATION, 505 SOUTH ROSA RD, MADISON, WI, 53719, US

(21) Appl. No.: 11/846,977

(22) Filed: Aug. 29, 2007

Related U.S. Application Data
(60) Provisional application No. 60/823,956, filed on Aug. 30, 2006.

(51) Int. Cl. A61K 39/00; A61P 35/00

(52) U.S. Cl. 424/184.1

(57) ABSTRACT

Proper timing and combinatorial administration of immunocytokines and Flt3-L combined with vaccination with tumor associated antigens provides an anticancer therapeutic benefit. Sequential administration of GM-CSF after Flt3-L provides improved expansion of mature dendritic cell populations. The expansion of mature dendritic cells can be used to enhance an immune response in the subject.

### CROSS-REFERENCE TO RELATED APPLICATIONS

This application claims the benefit of U.S. Provisional Application No. 60/823,956, filed Aug. 30, 2006.

### BACKGROUND OF THE INVENTION

The immune system of vertebrates consists of several interacting components. Two of the most important components are the humoral and cellular (cytolytic) branches. Antibody molecules, the effectors of humoral immunity, are secreted by special B lymphoid cells, B cells, in response to antigen. Antibodies can bind to and inactivate antigen directly (neutralizing antibodies) or activate other cells of the immune system, such as natural killer (NK) cells, to destroy the antigen or antigen presenting cell. Cellular immune recognition is mediated by a special class of lymphoid cells, the cytotoxic T cells or cytotoxic T lymphocytes (CTLs). These cells respond to peptide fragments, which appear on the surface of a target cell bound to major histocompatibility complex (MHC) proteins. The cellular immune system is constantly monitoring the proteins produced in all cells in the body in order to eliminate any cells producing foreign antigens. Humoral immunity is mainly directed at antigens which are exogenous to the animal, whereas the cellular system responds to antigens which are actively synthesized within the animal.

Cells of the immune system include antigen presenting cells, which process antigens and present them to other immune cells to stimulate one of the two pathways, helper T cells, T-effector lymphocytes, natural killer cells, polymorphonuclear leukocytes, macrophages, dendritic cells, basophils, neutrophils, eosinophils, monocytes.

The development of vaccines is frequently heralded as one of the most important medical breakthroughs. Prevention of disease has increased human life expectancy, lowered healthcare costs, and enhanced quality of life. Yet more widespread use is hampered by the difficulty in creating effective vaccines for new microbes and the expense associated with distribution and administration of current vaccines. Gene transfer can also be used as a vaccination and can address the problems associated with conventional vaccines. Vaccination can also be employed therapeutically. For instance, vaccinating a host for an antigen that is predominantly expressed on tumor cells is expected to elicit an immune response against this antigen and the tumor cells, thus eliminating these cells from the host.

When a foreign gene is transferred to a cell and expressed, the resultant protein is presented to the immune system. With a classic vaccine, the antigen itself is introduced into the host—either in the form of attenuated, killed or inactivated microbe, as purified (usually recombinant) protein, or as a synthesized peptide. With a genetic vaccine, the coding sequence for the antigen (or part of the antigen) is introduced into the host. Following transfection of the coding sequence into a host cell, the antigen is produced in situ. Expression of the antigen on the surface of a cell in the context of the major histocompatibility complex (MHC) results in an immune response. With genetic immunization, no protein purification or infectious agent preparation is necessary. Also with genetic immunization, truncations or added domains can be created by modification of the encoding polynucleotide. Finally, expression of a viral gene within a cell following gene delivery simulates a viral infection without the danger of an actual viral infection and induces a more effective immune response. This approach may be more effective in fighting latent viral infections such as human immunodeficiency virus, Herpes Simplex virus and cytomegalovirus.

Current genetic vaccination/immunization uses one of three methods: (1) direct injection of polynucleotide, such as naked DNA, into tissue such as skeletal muscle (optionally followed by electroporation); (2) ballistic delivery of plasmid DNA into the epidermis: gene gun (Chambers R S et al 2003); and (3) oral delivery of plasmid DNA (pDNA) formulations. Genetic vaccines have proven effective in eliciting immune responses against a wide variety of microbes. Protection in animal models has been demonstrated for influenza virus, malaria, bovine herpes virus, rabies virus, papilloma virus, herpes simplex virus, mycoplasma, lymphocytic choriomeningitis and others. The art has established that direct injection of pDNA into muscle is an efficient, reliable method for genetic vaccine delivery in mice. However, gene transfer following intramuscular injection of pDNA is less efficient in larger rodents and primates. The genetic vaccine trials have corroborated these earlier gene transfer and expression studies, by finding the need to inject large amounts of pDNA in human muscles to obtain good immune responses. Complexing pDNA with cationic liposomes (lipoplexes) has been attempted to enhance the efficiency of intramuscular and intranasal delivery.

Genetic vaccinations result in the induction of strong cytotoxic T lymphocyte (CTL) responses, where conventional subunit vaccines are skewed toward humoral responses (Donnelly J J et al. 1997; Pardoll D M et al. 1995). Since each individual genetic vaccine requires just the coding sequence for the antigen, many different vaccines can be produced and tested for each microbe. It is even feasible to generate a shot-gun library for a given microbe, vaccinate an appropriate animal model, and determine which clones result in the greatest immunity (either humoral or cellular). Alternatively, the expression of multiple epitopes allows genetic vaccines to better cover the variability in antigen presentation that exists in the population due to major histocompatibility (MHC) polymorphism. Because antigen expression has the potential to be maintained over a period of time, single dose immunization may also be possible with genetic immunization.

Genetic vaccines elicit both strong humoral and T cell responses, thus providing better memory activity against microbes such as malaria. The effectiveness of DNA vaccines to produce both humoral and cellular immunity indicates that DNA is expressed after administration, with the protein or peptide product being presented as an antigen in association with either Class I or Class II proteins. The immune response can be tailored by co-expression of cytokines. For instance, expression of IL-12 or interferon-γ skews the response toward Th1 whereas co-expression of IL-4 results in a Th2 type response. Th1 Helper T Cells are essential for controlling such intracellular pathogens as viruses and certain bacteria, e.g., Listeria and Mycobacterium tuberculosis (the bacillus that causes tuberculosis). Th2 Helper T Cells provide help for B cells and, in so doing, are essential for antibody-mediated immunity. Antibodies are needed to control extracellular pathogens. Many publications have recently shown the effects of co-expression of interleukins and other cytokines, which should allow for fine tuning of the immune response following administration of genetic vaccines. It has been hypothesized that transfer of antigen from myogenic cells to professional APCs can occur, thus obviating a requirement for direct transfection of bone marrow-derived cells (such as B-cells, T-cells, and APCs).

Antigen-presenting cells (APCs) regulate the development of immunity and tolerance. Dendritic cells (DCs) which are positive for the cell-surface molecule CD11c, the most potent APCs, play a central role in the presentation of antigen (Ag) to naïve T cells and in the induction of primary immune responses. They are primarily bone marrow-derived leukocytes that are widely distributed throughout the body in both lymphoid and non-lymphoid tissues and include epidermal Langerhans cells, splenic marginal zone DC, and interstitial DC within non-lymphoid tissues. DCs are typically located at sites of pathogen entry (the epidermis, mucosal epithelia, and the interstitial connective tissue of non-lymphoid organs) and acquire and process Ag from pathogens or pathogen-infected cells. Dendritic cells (DC) are of importance in immunophysiology: immunology, tolerance, HIV infection, cancer vaccines, and autoimmunity (Banchereau J et al. Nat Rev Immunol, 5: 296-306, 2005; Hackstein H et al. Trends Immunol, 22: 437-442, 2001; Larsson M. Springer Semin Immunopathol, 26: 309-328, 2005; Manfredi A A et al. Arthritis Rheum, 52: 11-15, 2005; Mellman I et al. Cell, 106: 255-258, 2001).

Two maturation states are distinguished for conventional DCs: immature and mature. Immature DCs display a phenotype reflecting their specialized function as Ag-capturing cells. When activated (i.e., triggered) by Ag (or DC modulation factors), tissue resident immature DCs undergo a differentiation process called maturation, into migratory and immunostimulatory active mature DCs (a terminally differentiated state). Immature DCs represent a heterogeneous population of cells that differ in the expression of pathogen recognition receptors (PRR) that are specialized for the capture of antigens from distinct pathogens. Mature DCs up-regulate their capacity to present captured Ag to T cells and induce CD4⁺ T cells and CD8⁺ cytotoxic T lymphocyte (CTL) responses. These mature DCs express high amounts of co-stimulatory molecules (CD80, CD86 and CD40) and cytokines (IL-12) and can initiate primary T-cell-dependent immune responses, including natural-killer-cell (NK) function. DCs also induce and regulate T cell tolerance within the peripheral lymphatic system. Upon presentation of antigen to T cells in the lymphoid organ, the maturation state of the DC controls the outcome of the immune response. Antigens taken up by immature DC in the steady state are presented in a tolerogenic manner. Immature DCs are considered inducers of T cell tolerance. Exposure to DC-modulation factors cause DCs to mature and change their expression of co-stimulatory and adhesion molecules, cytokine production, and migratory behavior.

A hallmark of DC maturation is the induction of CD83 surface expression (Cao et al. 2005). Maturation results in greater efficiency of Ag processing and presentation. Expression of co-stimulatory molecules, such as CD80, CD86 and CD40 by mature DCs is required for productive T cell stimulation (Liwksi et al. 2006). For DC-based strategies of immune activation, such as vaccines, CD83⁺ mature DCs have demonstrated a clear advantage over immature DCs in effectively inducing Ag-specific T cell responses.

DCs normally constitute less than one percent of blood mononuclear leukocytes. Moreover, >99% of spleen DCs are functionally and phenotypically immature and incapable of facilitating immune activation of T cells. Elaborate culturing systems requiring the timely addition of numerous recombinant cytokines and growth factors have been devised to expand DCs in vitro. Culturing blood mononuclear leukocytes in vitro, in the presence of granulocyte-monocyte colony stimulating factor (GM-CSF) and interleukin-4 (IL-4), has been shown to result in an expansion of a phenotypic and functional heterogeneous population of dendritic cells that were predominantly immature. DCs have been expanded in vivo by transplantation of tumors transduced with GM-CSF. Direct injection of GM-CSF has been less successful (Daro E et al. J Immunol, 165: 49-58, 2000). Injection of polyethylene glycol (PEG) modified GM-CSF resulted in expansion of myeloid-lineage (CD11c⁺, CD11b⁺) DCs in vivo (Pulendran B et al Proc Natl Acad Sci USA, 96: 1036-1041, 1999). In mice and humans, DCs can be generated in vivo and are distributed throughout the body by administration of the hemopoietic growth and differentiation factor, Fms-like tyrosine kinase ligand (Flt3-L) (Maraskovsky E et al. J Exp Med, 184: 1953-1962, 1996; Shurin M R et al. Cell Immunol, 179: 174-184, 1997; Maraskovsky et al. Blood, 96: 878-884, 2000). However, most of these are immature DCs of lymphoid-lineage (CD11c⁺/CD11b⁻). The administration of Flt3-L has also led to substantial increases in peripheral blood monocytes and circulating DCs, resulting in increased DCs at tumor sites as well as increased DCs available for leukapheresis and vaccine generation. Combined, simultaneous Flt3-L plus PEG-GM-CSF protein or gene therapy treatment has demonstrated increased CD11c⁺ DCs beyond that achieved by single agent delivery (Daro E et al. Cytokine, 17: 119-130, 2002; Daro E et al. J Immunol, 165: 49-58, 2000; Peretz Y et al. Mol Ther, 6: 407-414, 2002). Unfortunately, these DCs are also predominantly immature and require further ex vivo manipulation to attain an immunostimulatory mature DC phenotype.

The combination of signals such as IFNγ plus CD40-Ligand (CD40L, a cross-linking CD40 agonist) induced the production of high levels of IL12 from a subset of mature, in vitro-generated DCs that were more effective at inducing antitumor CTL responses in vitro (Mosca P J et al. Blood, 96: 3499-3504, 2000). Unexpectedly, it was found that DCs isolated from patients following Flt3-L treatment required an initial period of culture with GM-CSF prior to IFNγ plus CD40-L signaling in vitro to generate IL12-producing CD83⁺ DCs (48). Bone-marrow cells cultured concurrently with LPS (a microbial agent that can induce certain aspects of DC maturation) and GM-CSF also produced only immature DCs. Immature DCs from GM-CSF plus IL4 bone marrow cultures exhibited a greater development of IL12-producing CD83⁺ DCs when subsequently exposed to a maturation cytokine cocktail (TNFα, IL1b, IL6, and prostaglandin E₂) followed by CD40L (Kalady M F et al. J Surg Res, 116: 24-31, 2004). These studies indicated that temporal exposure of a series of signaling events to immature DCs in vitro can have effects on their maturation status and ability to be immunologically effective (Kalady M F et al. J Surg Res, 116: 24-31, 2004).

These and other methods produce DC populations that differ in terms of phenotype, cytokine secretion profile, ability to migrate to lymphoid compartments, and their interaction with T cells, all of which mediate a crucial role in eliciting a response that may be immunogenic or tolerogenic. Thus, there is a need to provide a stable supply of functionally and phenotypically characterized primary (non-immortalized) DCs for in vitro, ex vivo and in vivo studies. The present invention provides a method to generate DCs for research or therapeutic purposes, including: immune activation, vaccination, DC-based vaccines, cell biology, tolerance, antitumor therapy, organ transplantation, autoimmunity, and others. The invention may be applied to patients, in conjunction with a vaccination procedure, to induce immunity against infectious disease. As well, the invention can be applied to patients prior to autologous DC harvest to boost mature DC recovery for strategies that employ DC-based vaccines in the treatment of cancer.

Natural killer (NK) cell-mediated and T cell-mediated responses reflect complementary antitumor effector mechanisms against tumors (Algarra I et al. 2004, Bubenik J 2003). Immunotherapy has focused on either augmenting one response or the other. Approaches that rely predominantly on a single antitumor effector mechanism can favor development of tumor escape variants (TEVs), via immunoediting. Given their distinct antitumor mechanisms of action, we show that a combinatorial approach which includes concomitant NK- and T cell-dependent immune responses results in increased antitumor efficacy. A combinatorial approach using novel reagents and strategies to concurrently evoke NK- and T cell-mediated immunity is described. This combinatorial approach results in greater antitumor impact against established cancers, while preventing the development of residual refractory disease. In one embodiment, the combination of NK-dependent KS-IL2 immunocytokine treatment in conjunction with CTL-eliciting xenogeneic DNA vaccination against the universal tumor associated antigen (TAA) (UTA) telomerase reverse transcriptase (TERT) is used. Stimulating both the NK and T cell effector arms of this combinatorial approach can be done through gene therapy with Fms-like tyrosine kinase-3-ligand (Flt3-L).

Tumor escape. Naturally occurring innate (Smyth M J et al. 2002) and adaptive immunity can effectively eliminate many spontaneously arising subclinical tumors through immunosurveillance. However, this surveillance applies selective pressure to developing tumors to give rise to tumor cells that are resistant to immune recognition or destruction. This process is known as immunoediting and can lead to tumor escape from immunosurveillance with the eventual development of clinically detectable cancer (Dunn G P et al. 2004, Dunn G P et al. 2002). Accordingly, a combinatorial immunotherapeutic approach that simultaneously activates distinct antitumor effector mechanisms results in greater impact against established tumor and reduces the potential for metastatic disease or tumor recurrence. The leading cause of death in many cancer patients is metastatic burden. The combination of two distinct and complementary approaches (NK-mediated IC therapy and CTL-mediated cancer vaccines), each enhanced by Flt3-L gene therapy, provide improved antitumor benefit and TEV avoidance.

Universal tumor antigens: telomerase reverse transcriptase (TERT) and survivin. Vaccination with typical tumor-specific TAAs is expected to result in immunoselection of tumor cell variants with low or no TAA expression. By vaccinating against universal tumor antigens, such as TERT, whose expression is mandatory to maintain a malignant tumor phenotype, this problem in minimized. Nearly 90% of all human tumors exhibit TERT over-expression. In contrast, TERT shows restricted expression in normal tissue. Survivin is essentially absent in normal adult tissues. Expression is up-regulated in virtually all types of cancer studied, including ovarian cancer (Ambrosini G et al. 1998). We now show that simultaneous induction of CTL antitumor reactivity against TERT and survivin effectively target ovarian cancer cells exhibiting this highly malignant phenotype. Reduced TERT or survivin activity induces apoptotic death of cancer cells (Ambrosini G et al. 1998; Hahn W C et al. 1999; Jiang F et al. 2004; Ma X et al. 2005; Su Z et al. 2005; Vonderheide R H et al. 2004).

Regulatory T cells. An important mechanism of peripheral tolerance is active suppression by regulatory T cells (Tregs) (Wing K et al. 2005). These cells are primarily described as CD4⁺ CD25⁺ T cells that constitutively express CTLA-4 (cytotoxic T-lymphocyte-associated antigen-4), GITR (glucocorticoid-induced TNFR-related protein) and Foxp3 (a transcriptional regulator). Treg can inhibit immune responses to TAAs, thereby promoting tumor growth. In contrast, depletion of Treg can result in tumor resolution by increasing the activity of existing tumor-reactive CTLs. Recruitment of Tregs in the tumor microenvironment correlates with reduced survival in ovarian cancer patients (Curiel T J et al. 2004). The maturation state of DCs may be critical in the development of Tregs. Immature DCs (iDCs) promote their activation and expansion (Mahnke K et al. 2005). The genetic vaccination method described here induces the development of mature DCs (mDCs) at the site of vaccination prior to UTA expression. An increase in mature DCs limits the induction of Tregs and promotes greater development and activity of UTA-reactive CTLs.

Immunological tolerance and genetic cancer vaccines. Cancer cells arise from normal host cells that still utilize essentially the same cellular molecules as healthy cells. The immune system is tolerized against reacting to these ‘self’ antigens. Thus, it is difficult to generate an immune response against cancer cells. If T cells against these poorly immunogenic TAAs can somehow be activated through vaccination or other means, they can reject tumors presenting such self TAAs. A stable MHC/peptide complex is critical in the formation of the T cell and APC synapse (Slansky J E et al. 2000), and suggests that the mutated or altered-self antigen, may promote effective T cell activation by formation of a more durable synapse by a sustained MHC/altered-self antigen complex (Dyall R et al. 1998). Cancer vaccination using the altered-self concept has been extensively study against melanoma. A study involving the melanosomal TAA, gp100, demonstrated that a single amino acid substitution in a poorly immunogenic peptide resulted in conversion to a heteroclitic antigen that provided a 6-fold improved affinity for the MHC class 1 molecule, resulting in activation of naïve T cells and effectively breaking tolerance (Yu Z et al. 2004). In a similar fashion, expression of altered-self TAAs derived from orthologs of different (xenogeneic) species may help break tolerance. We have shown that hydrodynamic intravascular delivery of full-length human gp100 (hgp100) DNA is able to elicit prophylactic protection against B16 tumors transfected to express the hgp100 xenoantigen (B16-hgp100). More importantly, this vaccination procedure also breaks tolerance by providing protection to hgp100-vaccinated mice challenged with the B16 tumor expressing the murine-gp100 self TAA (Neal et. al.).

Genetic vaccination permits proper post-translational processing of the antigen gene products and presentation that is compatible with the differing individual host's MHC haplotypes (Howarth M et al. 2004). The described genetic vaccination strategy facilitated breaking immune tolerance and inducing meaningful antitumor CTL activity against TERT and survivin without the need to predetermine any specific immunogeneic regions within these two UTA molecules.

### SUMMARY OF THE INVENTION

The present invention provides methods for the treatment of cancer by stimulation of the immune system. Several anti-cancer treatment methods are described which, alone or in combination, provide enhanced therapy, minimize the development of tumor escape variants, and result in anti-tumor memory. These treatments include:
A. Flt3-L gene therapy, which allows for better delivery of this biological, avoiding problems associated with Flt3-L protein therapy and induces a greatly expanded pool of NK cells. The expanded pool of NK cells may be used to enhance the effectiveness of immunocytokine therapy.
B. Flt3-L plus secondary factor gene therapy to greatly expand the number of mature dendritic cells, which improves the effectiveness of cancer vaccines.
C. The use of universal TAAs, which limit TEV development.
D. Vaccination following Flt3-L with or without secondary factor gene therapy to provide long lasting immunity.
E. The combination of Flt3-L gene therapy with or without secondary factor therapy and IC therapy to provide a combinatorial treatment invoking multiple immune system pathways resulting in improved tumor destruction and immune memory.

In a preferred embodiment, therapy includes the in vivo expansion of an immune cell population in mammals. A preferred immune cell population consists of dendritic cells. More preferably, the immune cell population consists of mature dendritic cells. This method is comprised of sequential in vivo administration of several DC-modulation factors. In a preferred embodiment, these factors include Flt3-L, GM-CSF and CD40-L. These factors can be administered as an effective amount of protein or modified protein. Alternatively, genes encoding these DC-modulation factors can be delivered to cells in the mammal where they are expressed. Delivery of a gene provides for continuous in vivo exposure of the proteins over several days to weeks. Any known effective gene delivery method may by used to delivery these genes to cells in vivo. Exemplary gene delivery methods include hydrodynamic injection of naked DNA, direct injection of DNA and viral and non-viral vectors.

In a preferred embodiment, the in vivo expansion of dendritic cells comprises: sequential delivery of Flt3-L, followed by other cytokines or growth factors (secondary factors). The timing of delivery of the secondary factor(s) relative to the delivery of Flt3-L influences the effect of the secondary factor on immune cell expansion both quantitatively and qualitatively. These secondary factors result in further expansion, and more importantly, “maturation” of the Flt3-L expanded population of immature dendritic cells. In a preferred embodiment, the secondary factor(s) are delivered around the time of maximal dendritic cell expansion induced by Flt3-L. In a preferred embodiment, the secondary molecule is GM-CSF and/or CD40-L (also known as CD154). Critical to this invention and to the ability of the secondary factor-induced maturation process of the pool of Flt3-L-expanded immature DCs, is the continuous in vivo exposure of an effective dose of the secondary factor(s). When delivered as unmodified protein, GM-CSF has a terminal half-life of 1 hr and is likely responsible for the poor performance of GM-CSF protein administration in its ability to expand and mature DCs. In a preferred embodiment, these DC-modulating factors are administered in vivo in a fashion that provides continuous, effective exposure to facilitate the transition of immature DCs to a functionally mature DC phenotype.

In a preferred embodiment, the expanded population of immune cells can be collected from blood, lymph nodes, spleen or bone marrow of the mammal and used for research, diagnostic or therapeutic purposes. Additionally, this invention can be utilized in the clinical setting as a mean to expand and mature autologous patient DCs to enhance therapeutic approaches such as greater cancer vaccine efficacy. The ability to expand and mature DCs within a patient, without the need for any ex vivo manipulation of patient cells, is an important and key clinical application of this invention.

In a preferred embodiment, the expanded population of immune cells is used to improve tumor therapy. Expansion of dendritic cells, in particular mature dendritic cells, provides a source of antigen presenting cells. Thus, delivery of an antigen in a host primed with Flt3-L and optionally secondary factors (e.g., GM-CSF) results in a more vigorous and effective anti-antigen immune response. A preferred antigen is a tumor specific antigen. Another preferred antigen is a universal tumor antigen (UTA), such as TERT, survivin or livin, whose expression is obligatory to maintain a malignant tumor phenotype.

In a preferred embodiment, Flt3-L is administered to expand the population of natural killer cells and improve subsequent immunocytokine therapy. Immunocytokines partly mediate their action via the NK-mediated process of Antibody-Dependent Cell-Mediated Cytotoxicity (ADCC). Thus, expansion of the NK cell population increases the effectiveness of immunocytokine anti-tumor therapy. In a preferred embodiment, immune cell expansion mediated by Flt3-L is combined with immunocytokine treatment and tumor antigen vaccination. This anti-tumor therapy enlists multiple immune pathways and improves anti-tumor therapy. In a preferred embodiment, the combination therapy destroys tumors cells via ADCC and CTL-mediated processes and creates an immune memory capable of suppressing tumor reemergence.

### BRIEF DESCRIPTION OF THE DRAWINGS

FIG. 1. HLV Flt3-L plus IC effect. Groups (n=4) of A/J mice were injected with NXS2 cell i.d. on d 0 and received (A) no treatment, (B) 200 μg mFlt3-L DNA by HLV d 3, (C) 10 μg/d hu14.18-IL2IC (d 11-14), or (D) both Flt3-L+IC. Arrows indicate when treatment was given. (E) Mice that had received Flt3-L+IC were re-challenged with NXS2 tumor 70 d later and compared to (F) naïve mice for tumor progression.

FIG. 2. Confocal images of muscle sections stained with CD11c (1 and 2), with CD83 (3), with isotype IgG (4). Top row (A-D) shows images in red channel visualizing targeted antigens, bottom row shows the same images combined with nuclear (ToPro-3) and Actin (Phalloidin Alexa 488) staining. A,E-CD11c⁺ cell distribution in untreated limb muscle, B,F-CD11c⁺ cell distribution in limbs 4 d following sequential transfection with Flt3-L (d0) and GM-CSF (d10) by HLV delivery, (C,G)-CD83⁺ cell distribution in Flt3-L/GMCSF HLV treated limb, D,H-negative control immunostaining with isotype Ab in Flt3-L/GMCSF HLV treated limb. Images G and H contain holes in muscle fibers, a freezing artifact often unavoidable in muscle cryosectioning. All images were taken under magnification x630.

FIG. 3. Anti-luciferase humoral response. Groups of ICR mice received genetic immunization of 50 μg of pCI-luciferase or pCI-hgp100 DNA by HLV injection on d10. Some groups also receive sequential Flt3-L/GMCSF gene delivery by HTV (10 μg pORF-mFlt3L d0, 2 μg pORF9-mGMCSF d10) or HLV (150 μg pORF-mFlt3L d0, 50 μg pORF9-mGMCSF d10) injection beginning on d0. Specific combinations of sequential Flt3-L/GMCSF delivery plus genetic immunizations are indicated below the figure. Group pooled sera (n=3) collected on d20 (d10 post-genetic immunization) and d34 (d24 post genetic-immunization) was assessed for mouse anti-luciferase antibody using a standard ELISA procedure. A standard curve was developed using a commercially available primary mouse-anti-luciferase monoclonal antibody. Data reported as mean values (μg/ml) for group-pooled sera collected on a specific date.

FIG. 4. Detection of gp100-specific T cells following combined Flt3-L/GM-CSF plus hgp100 HLV vaccination. PBMCs were assessed for the presence of double positive cells staining for murine CD8 and the hgp100₂₅₋₃₃/H2D^{b}-tetramer. Groups (n=3) of mice received a single HLV delivery of 50 μg of hgp100 pDNA in both the right and left hind-limbs on d10 (B) or 200 μg Flt3-L pDNA on d0 and 50 μg of GM-CSF+50 μg of hgp100 pDNA (mixed) in both right and left hind-limbs on d10 (C). Group-pooled blood was depleted of erythrocytes by hypotonic shock 10 d following final vaccination. PBMCs were labeled with anti-murine CD8-FITC (BD Biosciences, San Jose, Calif.) and hgp100₂₅₋₃₃/H2D^{b} tetramer-PE (Beckman Coulter Immunomics, San Diego, Calif.) and analyzed by flow cytometry. Negative control sample is from naïve mice (A). Cells were 2-color stained for murine CD8-FITC and hgp100₂₅₋₃₃/H2D^{b}-tetramer-PE and analyzed by flow cytometry on d14.

FIG. 5. Antitumor response in combinatorial vaccinated mice. Groups (n=4) of naïve mice were HLV vaccinated by HLV delivery of 50 μg of hgp100 pDNA in the right hind limb on d10 (circle) or combinatorial vaccinated (triangle) by HLV delivery of 200 μg of Flt3-L pDNA on d0 followed by 50 μg of GM-CSF+50 μg of hgp100 pDNA (mixed and concurrently injected) on d10. Mice were tumor challenged with 1×10⁶ B16 tumor cells on d20. Naïve control mice were also challenged (square). Tumor growth for each treatment group is represented by group-mean tumor volume±SE

FIG. 6. Example treatment timeline.

FIG. 7. shows Flt3-L (FL) plus IC effect on NXS2 tumor growth. Groups (n=4) of A/J mice were injected with 5×10⁶ NXS2 cell i.d. on d 0 and received (A) no treatment, (B) 200 μg mFL DNA by HLV in both hind limbs on d 3, (C) 10 μg/d hu14.18-IL-2 IC (d 11-14), or (D) both FL DNA+IC. Small (FL) and large (IC) arrows indicate HLV gene delivery and hu14.18-IL-2 treatment initiation dates, respectively. (F) Mice that had received FL+IC (i.e., same animals use in panel D) were rechallenged with NXS2 tumor 70 d later and compared to (E) naïve mice for tumor progression. Data represents NXS2 tumor growth for individual animals.

FIG. 8. Modulation of MHC class I expression. Groups of A/J mice (6 mice in group A and 8 mice in all other groups) were injected with NXS2 cell i.d. on d 0 and received (A) no treatment, (B) 200 μg mFL DNA by HLV in both limbs on d 5, (C) 200 μg mFLex DNA by HLV in both limbs on d 5, (D) 10 μg/d hu14.18-IL-2IC (d 12-15), (E) both FL DNA+IC, or (F) both FLex DNA+IC. Data represents NXS2 tumor growth for individual animals. Primary tumors were harvested on d 27 following NXS2 cell injection and individually profiled for H2D^{d} expression. The specific MFI ratio for H2D^{d} expression is represented numerically and is adjacent to tumor growth graph for that particular tumor lesion. Due to progressive tumor growth, some mice were euthanized prior to the tumor harvest date. Small (FL) and large (IC) arrows indicate HLV gene delivery and hu14.18-IL-2 treatment initiation dates, respectively.

### DETAILED DESCRIPTION

We have developed improved methods for cancer therapy. Delivery of Fms-like tyrosine kinase ligand (Flt3-Ligand or Flt3-L) results in an expansion of natural killer (NK) cells. In a preferred embodiment, an immunocytokine (IC) is administered to a patient after Flt3-L treatment has expanded the NK cell population. The therapeutic effects of ICs are in a large mediated via NK cells. Thus, the therapeutic effects of ICs are improved following expansion of the NK effector cell population.

Sequential delivery of Flt3-L and granulocyte-macrophage colony-stimulating factor (GM-CSF) promotes expansion and maturation of DCs capable of Ag-specific T cell activation. In a preferred embodiment, CD40-Ligand (CD40-L) is also administered concurrently or subsequent to GM-CSF delivery, and provides an additional, but distinct, signaling response to facilitate further maturation of the Flt3-L-generated immature DCs.

Delivery of Flt3-L, GM-CSF and CD40-L can be by injection of purified proteins, injection of modified proteins, delivery of DNA encoding these proteins, or a combination of these. Modified proteins include, but are not limited to, PEG or similar modified forms of the proteins, and amino acids variants which retain activity but have longer half-lives, increase activity, or are less immunogenic. DNA encoding these factors can be delivered by any known gene delivery methods, including viral and non-viral vectors. The DNA can be delivered to liver, skin, skeletal muscle or any tissue which is able to express and secrete an active form of the protein. To minimize unintended immune responses, species-specific factors are used (i.e., use of the invention in mice would preferentially employ delivery of murine-derived factors). In one embodiment, these DC-modulation factors are administered in a fashion that affords a continuous in vivo exposure of physiologically relevant and effective levels of these factors during the treatment time period. For delivery of purified protein, continuous administration may mean giving the subject multiple or continuous doses of the protein over days or weeks. For delivery of a transgene, continuous administration is accomplished by continued expression of the transgene from the transfected cell. Various promoters readily available in the art may be used to drive expression of the transgene.

Flt3-L and GM-CSF have previously been shown to aid in expanding DC populations in vivo. When given together concurrently, these factors act additively to further expand DC populations. Unfortunately, even with this combined treatment, the DCs have been predominantly immature and require further ex vivo manipulation to attain a mature DC population with an immunostimulatory phenotype. We now show that sequentially administering first Flt3-L followed by subsequent GM-CSF delivery, results in a more pronounced expansion of splenic CD11c⁺ DCs that further exhibit the desired mature phenotype. These cells are phenotypically and functionally mature DCs capable of Ag-specific T cell activation. Inclusion of CD40-L administration as a third component of this multifactor regimen drives the maturation process further, resulting in a greater frequency and absolute number of DC having the desired mature phenotype and function. Moreover, the described methods provide a clinically applicable means to dramatically and significantly increase the number of mature DCs in a patient without the need for ex vivo cell manipulation. By combining the DC expansion process with the delivery of a tumor antigen (TAA), the patient's immune system can be activated to specifically target tumor cells.

We show that, in mice, sequential delivery of plasmid DNA (pDNA) vectors coding for murine Flt3-L followed by murine GM-CSF (mGM-CSF) results in a >1000-fold in vivo expansion of splenic DCs displaying a mature phenotype. In ICR mice, sequential gene delivery, such as by hydrodynamic tail vein injection (HTV), of 10-20 μg of Flt3-L pDNA followed by 2-5 μg of GM-CSF pDNA 10 days later resulted in a >10-fold increase in spleen cellularity and a >1000-fold increase in mature DCs when harvested on day 14. Inclusion of CD40-L, delivered as a pDNA expression vector concurrently with GM-CSF pDNA or subsequent to GM-CSF pDNA, may promote even greater expansion and development of mature DCs.

We show that by delaying GM-CSF treatment until Flt3-L treatment has already initiated immature DC population expansion, the numbers of DCs are further increased and that the percentage of mature DCs is greatly increased. Providing an interval between Flt3-L treatment and GM-CSF treatment allows Flt3-L to result in an expanded population of immature DCs which are then subsequently exposed to GM-CSF. Depending on the route of Flt3-L administration (as protein or by gene delivery), maximal immature DC expansion may occur 5-15 days following initiation of Flt3-L treatment. A preferred dose of Flt3-L is the minimum amount that induces a maximum expansion of immature DCs preferably about 5 to about 15 days, and more preferably about 8 to about 12 days, and more preferably about day 10, following initiation of Flt3-L treatment. The Flt3-L dose at rate of DC expansion may be different for different species. However, these values are readily determined using methods standard in the art. Preferably, GM-CSF is administered within about 2 days of maximal immature DC expansion following initial Flt3-L administration. In mice, GM-CSF treatment about 10 days after Flt3-L administration resulted in dramatically increased mature DC populations. The desired dose of GM-CSF is the minimum amount that promotes the greatest frequency and absolute number of mature DCs, while minimizing the collateral development of GM-CSF-induced myeloid suppressor cells. This value may be different for different species and is readily determined. Concurrently delivering CD40-L with GM-CSF uses a dose that provides maximal mature DC-expansion at a minimum dose. For subsequent delivery to GM-CSF, the timing of CD40-L administration is determined empirically on a species-specific basis.

The described invention results in dramatic and substantial in vivo expansion in the frequency and absolute number of mature DCs. These cells are phenotypically and functionally mature DCs capable of Ag-specific T cell activation. Moreover, when used with research animals, the resulting mature DCs are in sufficient numbers and can be harvested, cryopreserved, and redistributed to the scientific community. The fraction of immature DCs can also be harvested, isolated and distributed. The harvested DCs can be utilized as a renewable source of primary-mature DCs for areas of research and translational studies including: immune activation, vaccination, DC-based vaccines, cell biology, tolerance, antitumor therapy, organ transplantation, autoimmunity, and others.

The invention can be used as a method to expand mature DCs in vivo in research animals for basic and translational research that seeks to boost immune activation or responsiveness. These efforts may include investigations in development of novel cancer vaccine strategies, breaking immune tolerance, control or prevention of infectious disease, treatment of HIV/AIDS and other therapeutics that involve immature DCs/mature DCs.

By increasing the dose of GM-CSF relative to the Flt3-L dose, the in vivo response can be skewed toward intentional expansion of myeloid suppressor cells (CD11b⁺/Ly6G⁺ granulocytes). These suppressor cells are well characterized for their ability to mediate immunosuppressive activity. Accordingly, harvested or in vivo expansion of myeloid suppressor cells could be utilized in preclinical efforts that seek to suppress aspects of immunity such as: organ transplant immunotherapy, and the treatment of autoimmune diseases like lupus, rheumatoid arthritis and Wegener's granulamatosis.

In the clinical setting, this invention can be used to expand autologous mature DCs in patients undergoing immunotherapy. As an example, for DC-based vaccine strategies, the invention would expand mature DCs in patients prior to DC harvest. These DCs are then manipulated ex vivo to create a DC-vaccine, and given back to the patient. The invention can be used to expand mature DCs in vivo and be used as a component of a vaccination regimen in the treatment of cancer or infectious disease. The invention may be used to better vaccinate for greater prophylactic immunity. Preferred tumor vaccination antigens include those specifically associated with tumors, such as gp100, MARTI and NY-ESO-1. Many such tumor associated antigens are known in the art. Preferred antigens are universal antigens. These are associated with functions required for cell survival. Thus, down regulation of expression of the antigen is not a viable escape strategy for the tumor cells.

This invention can be used to expand NK cells in patients. Therapeutics that mediated their effect through NK cells would benefit from such a pretreatment. We show that the therapeutic effects of IC treatment are significantly enhanced by pretreatment with Flt3-L. A combination therapy of Flt3-L, a secondary factor (preferably GM-CSF), a tumor antigen and an immunocytokine would provide a multi-pronged antitumor approach. This treatment would activate multiple immune pathways (NK-mediated, CTL-mediated) and provide short term anti-tumor cell activity, as well as long term anti-tumor cell memory.

The invention can be applied to aid in reconstitution of the immune system and function following ablative therapies that induce immunosuppression, such as encountered following treatment with various chemotherapeutics.

As similarly indicated for basic and preclinical research application, the dosing of Flt3-L and GM-CSF can be modified to intentionally induce the development of myeloid suppressor cells in patients. Development of these suppressor cells can be instrumental in therapeutic efforts to control organ rejection in recipients, and in the treatment of autoimmune disorders.

Recent evidence suggests that amelioration of the symptoms and conditions associated with Crohn's disease (an inflammatory bowel disease) is achieved with daily treatment of GM-CSF (Korzenik J R et al. N Engl J. Med. 352:2193-201 2005). Daily treatment is required due to the relatively short half-life of GM-CSF in vivo. As hydrodynamic gene delivery of GM-CSF expressing pDNA results in biologically relevant expression of GM-CSF for several days, hydrodynamic limb vein (HLV) of GM-CSF pDNA could be therapeutic in the treatment of Crohn's disease.

Fms-like tyrosine kinase 3 ligand (Flt3-L): FLT3 is a receptor tyrosine kinase (RTK) expressed by immature hematopoietic progenitor cells. The ligand for FLT3 (Flt3-L) is a transmembrane or soluble protein and is expressed by a variety of cells including hematopoietic and marrow stromal cells; in combination with other growth factors, the ligand stimulates proliferation and development of various cell types including: stem cells, myeloid and lymphoid progenitor cells, DCs and NK cells. Activation of the receptor leads to tyrosine phosphorylation of various key adaptor proteins known to be involved in different signal transduction pathways that control proliferation, survival and other processes in hematopoietic cells. As used herein, the term Flt3-L refers to polypeptides that bind Flt3 receptor found on progenitor and stem cells and possess biological activity.

Granulocyte macrophage colony-stimulating factor (GM-CSF): GM-CSF is a protein secreted by macrophages that stimulates stem cells to produce granulocytes (neutrophils, eosinophils, and basophils) and macrophages. It is thus part of the immune/inflammatory cascade, whereby activation of a small number of macrophages produces more of them in circulation. GM-CSF is distinct from granulocyte colony-stimulating factor (G-CSF). Additionally, Activated CD4⁺, CD8⁺ T cells, NK and DCs all secrete GM-CSF.

Although GM-CSF has long been considered an immune adjuvant, recent evidence underlies its dual role in stimulating as well as suppressing the immune system. Many human and murine tumor cells lines (including breast, cervical ovarian, prostate, colon, renal cancer as well as melanoma) secreted this cytokine. Moreover, GM-CSF secretion by various transplantable mouse tumors has correlated with the capacity to metastasize. As well, administration of GM-CSF protein in mice is sufficient to recruit myeloid suppressor cells into the secondary lymphoid organs and suppress antigen-specific CD8⁺ T cell responses.

On the other hand, GM-CSF has been shown to elicit powerful immune responses when combined with irradiated tumor cell vaccines, in various murine models and in the clinical setting, which has lead to its widespread use as an immune adjuvant to augment antitumor immunity. The amount of GM-CSF administered is critical in determining the ensuing immune response. A relatively low dose of GM-CSF in a vaccine formulation enhances immunity, while higher doses result in significant in vivo immunosuppression mediated by myeloid suppressor cells recruitment. These findings support the dual role of GM-CSF on the immune response.

GM-CSF can promote expansion of DCs. Unlike Flt3-L, GM-CSF also acts to recruit DCs locally to the site of administration, and facilitates some aspects of DC maturation.

CD40-L (also known as CD154): CD40-L is a member of the tumor necrosis factor (TNF) family of cell surface interaction molecules. It is a 261-amino-acid type II membrane glycoprotein, and its expression is mainly confined to the CD4⁺ T cell subset. CD40L expression is induced shortly after T-cell activation and represents an early activation marker of T lymphocytes. CD40 is expressed mainly on B cells, macrophages, and DCs. The CD40-CD40L pathway has been extensively investigated and has been shown to play multiple functional roles in the healthy immune system. It enhances the antigen-specific T-cell response through the activation of DCs and the induction of interleukin 12 (IL-12) production by these cells to focus the immune response on the antigen that has engaged the TCR. Activation of APC by CD40-CD40L interaction induces the production of inflammatory cytokines, chemokines, nitric oxide, and metalloproteinases. Interaction of CD4⁺ CD40L⁺ T cells with CD40 on B cells leads to B-cell differentiation, proliferation, immunoglobulin (Ig) isotype switching, and formation of memory B cells.

CD40 expression by DCs is up-regulated when they migrate from the periphery to draining lymph nodes (DLN) in response to maturation signals. CD40-L signaling by MHC-restricted, activated CD4⁺ T cells induces differentiation of DC, as defined by an increased surface expression of MHC, co-stimulatory, and adhesion molecules. Thus, CD40 functions in the adaptive immune response as a trigger for the expression of co-stimulatory molecules for efficient T-cell activation. CD40 ligation of DC also has the capacity to induce high levels of the cytokine IL-12, which polarizes CD4⁺ T cells toward a T helper 1 (Th1) type, enhances proliferation of CD8⁺ T cells, and activates NK cells. CD40 may also play an important role in the decision between tolerance and immunity and the generation of regulatory CD4⁺ T cells that are thought to maintain peripheral self-tolerance in vivo.

An antigen-presenting cell (APC) is a cell that displays foreign and self antigen complexed with MHC on its surface. T-cells may recognize this MHC/antigen complex using their T-cell receptor (TCR). Although almost every cell in the body is technically an APC since it can present antigen to CD8⁺ T cells via MHC class 1 molecules, the term is often limited to those specialized cells that can prime T cells (i.e., activate a naïve T cell). These cells generally express MHC class II as well as MHC class I molecules, and can stimulate CD4⁺ (helper) T cells as well as CD8⁺ (cytotoxic) T cells. To help distinguish between the two types of APCs, those that express MHC class II molecules are often called professional antigen-presenting cells and generally refers to cells such as macrophages, B-cells and dendritic cells. These professional APCs are very efficient at phagocytosis, which allows them to present exogenous as well as internally derived antigens. For the purpose of effectively stimulating naïve T cells, APCs possess co-stimulatory molecules: cell-surface molecules that deliver essential signals to T cells, allowing the T cells to become activated and mature into fully-functional forms. Well known co-stimulatory molecules on APCs include CD80, CD86 and CD40.

Dendritic cells are bone marrow-derived cells that can be found in all lymphoid, and almost all non-lymphoid, tissues. DCs were discovered by Ralph Steinman and Zanvil Cohn more than three decades ago (Steinman, R. M. and Cohn, Z. A. J Exp Med, 137: 1142-1162, 1973.). DCs in different tissues may differ from each other with regard to function as well as phenotype. Surface expression of CD11c is considered a phenotypic hallmark of the vast majority of DCs Over the past decade, the discovery of a network of APCs that regulate the development of immunity and tolerance has provided insight into the complex relationships within the immune system. DCs, the most potent APC, play a central role in the presentation of Ags to naïve T cells (exhibit a >100-fold greater capacity to stimulate naïve T cells as compared to other professional APCs) and in the induction of primary immune responses. DCs are typically located at sites of pathogen entry and are uniquely efficient to acquire Ag from pathogens or pathogen-infected cells, transport Ag from the periphery to lymphoid tissues, and to process Ag for both Major-Histocompatibility-Complex (MHC) class I and class II presentation by cross-priming. Upon Ag encounter, the tissue resident immature DCs, undergo a terminal differentiation process called maturation. In addition to their obligatory role as initiators of primary adaptive immunity, DCs are now also seen as crucial regulators of aspects of innate immunity, in particular natural-killer-cell (NK) function. Aside from their well know immunostimulatory properties, DCs can also induce and regulate T cell tolerance in the periphery by presenting self-antigens (molecules expressed by normal tissue). In some instances, presentation of self-antigen can lead to immune stimulation and the development of autoimmune pathology, such as type 1 diabetes. A hallmark of DCs is surface expression of CD11c. DCs have been phenotypically described as myeloid-like (CD11c⁺CD11b⁺CD8⁻) DCs, lymphoid-like (CD11c⁺CD11b⁻CD8⁺) DCs, and plasmacytoid precursor DCs (CD11c⁺CD11b⁻CD8⁻). Some tissue-resident DCs are uniquely identified such as Kupffer cells (liver) or Langerhans cells (skin).

Immature DC: The DCs located in peripheral tissues in the immune steady-state have characteristics which make them ideally suited to monitor their environment for pathogens and to facilitate their uptake. They are said to be ‘immature’ and express a large array of receptors that can specifically recognize pathogen-related molecules. These include Toll-like receptor (TLR), which have specific recognition for a range of molecules including CpG DNA and lipopolysaccharides (LPS). Once in contact with antigen, immature DCs use several pathways to facilitate uptake. These include specific uptake by receptor-mediated endocytosis, and non-specifically through phagocytosis and macropinocytosis. Although many of these pathways appear to be utilized for uptake of pathogen-related molecules they may also be used for uptake of self antigens. Indeed, immature DCs also express (alpha)v(beta)3- and (alpha)v(beta)5-integrins that facilitate continuous uptake of apoptotic material in the immune steady state. Once in the endocytic pathway, internalized antigens must be processed before they can be displayed to lymphocytes in association with MHC molecules. Intracellularly derived or extracellularly acquired antigen can be processed and complexed with MHC class I and MHC class II molecules in a process termed ‘cross presentation’. However, before DC can complex processed antigen on to MHC molecules and display them at the cell surface, they must first undergo functional maturation. Immature DCs do not express CD83 and have low or absent expression of costimulatory molecules CD80, CD86 and CD40.

Maturation: The two well-established maturation states for DCs include the immature and mature states. Under conditions of infection or inflammation, DCs encounter signals such as pro-inflammatory cytokines and bacterial or viral products such as LPS, CpG motifs, and double-stranded RNA. These factors may induce the maturation of DCs, allowing DCs to present Ags in a manner that stimulates Ag-specific immunity. Activated DCs can be distinguished by expression of higher levels of MHC and costimulatory molecules or by production of cytokines such as IL12. Induction of IL12 by DCs appears to involve a multi-step process that requires ligation of the DC-expressed CD40 cell-surface molecule. Maturation can be induced in vitro by exposing bone-marrow precursor cells or immature DCs to cytokine/growth factor cocktails; with GM-CSF being a common component of all such culturing methods.

Mature DC: One of the first properties attained by ‘maturing DC’ is the capacity to migrate from non-lymphoid peripheral organs through afferent lymph to the T cell-rich paracortical areas of the proximal secondary lymphoid tissue. Maturing DCs also upregulate CCR-7 to enable maturing DCs to migrate towards lymphatic endothelium and to concentrate within T cell-rich areas. The maturing DC is also characterized by tight control over the formation of MHC/peptide complexes and their expression on the cell surface (i.e., increased half-life of surface-expressed MHC-peptide complexes) along with costimulatory molecules (CD80, CD86 and CD40). Internalized protein antigen can accumulate for extended time periods in immature DC. However, within 3-4 hr after induction of maturation of DC, antigen rapidly begins to complex with MHC molecules and is transported to the cell surface to enable Ag-presentation to T cells. Maturing DCs down regulate their endocytic capacity, thereby preventing re-absorption and degradation of MHC/peptide complexes and promoting their stable expression at the cell surface. MHC molecules are expressed 10 to 100-fold higher on mature DC than on B cells and macrophages. Mature DC also upregulate expression of several costimulatory molecules including CD80, CD86 and CD40 and also begin expression of a novel chemokine, DC-CK1, which preferentially attracts naïve (CD45RA⁺) T cells. CD4⁺ T cells respond by increasing surface expression of CD40-L, which can in turn interact with CD40 on mature DC empowering them to directly stimulate naïve CD8⁺ T cells. This bypasses the need for direct spatial interaction of CD8⁺ T cells with CD4⁺ T helper-1 cells. A definitive hallmark of mature DCs is surface expression of CD83.

T cells are a type of white blood cell that are central in cell-mediated immunity. There are several types of T cells including: Cytotoxic T cells, Helper T cells, and Regulatory T cells. T cells express T cell receptor (TCR) molecules on their surface, a heterodimeric receptor molecule that gives antigen-specificity to an individual T cell clone. Cytotoxic T cells (CTLs), also known as CD8⁺ T cells, destroy virally infected cells and tumor cells and are implicated in transplant rejection. Helper T cells, or CD4⁺ T cells, are the “middlemen” of the immune response. Once activated, helper T cells divide rapidly and secrete cytokines that regulate or help the immune response. CD4 and CD8 refer to characteristic glycoproteins on the surface of certain T cells. These CD molecules are convenient diagnostic markers for identifying T cells. Regulatory T cells (Tregs), formerly known as suppressor T cells, are crucial for the maintenance of immunological tolerance.

A two-signal mechanism is believed to be involved in activation of the different T cells types. Interaction between TCR molecules and specific MHC/antigen complexes on APCs delivers a signal into the T cell. Then, co-stimulatory interactions between CD28 molecules on the T cell and B7 molecules (i.e., CD80 and CD86) on the APC deliver a second signal, activating the T cell. Without co-stimulation a T cell becomes functionally inert (anergic).

Antibody-Dependent Cell-Mediated Cytotoxicity (ADCC) is a mechanism of cell-mediated immunity whereby an effector cell of the immune system actively lyses a target cell that has been bound by specific antibodies. It is one of the mechanisms through which antibodies, as part of the humoral immune response, can act to limit and contain infection. Classical ADCC is mediated by natural killer (NK) cells; monocytes and eosinophils can also mediate ADCC. ADCC is part of the adaptive immune response due to its dependence on a prior antibody response. Typical ADCC involves activation of NK cells and is dependent on the recognition of antibody-coated infected cells by Fc receptors on the surface of the NK cell. The Fc receptors recognize the Fc (constant) portion of antibodies such as IgG, which bind to the surface of a pathogen-infected target cell. The most common Fc receptor that exists on the surface of NK Cell is called CD16 or FcγRIII. Once bound to the Fc receptor of IgG the Natural Killer cell releases cytokines such as IFNγ, and cytotoxic granules containing perforin and granzymes that enter the target cell and promote cell death by triggering apoptosis.

Natural killer (NK) cells are a form of cytotoxic lymphocyte, which constitute a major component of the innate immune system. NK cells play a major role in the host-rejection of both tumors and virally infected cells. They are large granular lymphocytes that do not express TCR. NK cells are cytotoxic and can target self cells with low levels of MHC class I cell surface marker molecules. NK cells are activated in response to interferons or macrophage-derived cytokines.

The term antigen is well understood in the art and includes substances which induce and immune response. An antigen also refers to any agent that is recognized by an antibody or antibodies or binds to antigen receptors. The term antigenic determinant, or epitope, refers to a site on an antigenic molecule which binds to an antibody or specific receptor site on the sensitized lymphocyte. Thus, a single peptide, or antigen, can possess one or more antigenic determinants. The term immunogen refers to any agent that can elicit an immunological response in an animal. In many cases, antigens are also immunogens, thus the term antigen is often used interchangeably with the term immunogen. These terms may be used to refer to an individual macromolecule or to a homogeneous or heterogeneous population of antigenic molecules. A hapten is a substance that reacts selectively with appropriate antibodies or T cells but the hapten itself is usually not immunogenic. Most haptens are small molecules or small parts of large molecules, but some macromolecules can also function as haptens. Antigens can be presented by the cell in the context of the major histocompatibility antigens.

Antigens can be naturally occurring of they can be made by synthetic or recombinant methods. Antigenic peptides can be modified during or after translation, e.g., by phosphorylation, glycosylation, cross-linking, acylation, proteolytic cleavage, linkage to an antibody molecule, membrane molecule or other ligand (Ferguson et al. 1988). A self-antigen is an antigenic peptide that induces little or no immune response in the subject due to self tolerance to the antigen. The term tumor associated antigen or TAA refers to an antigen that is associated with or specific to a tumor. Examples of known TAAs include gp100, MART and MAGE.

The major histocompatibility complex (MHC) refers to a complex of genes encoding cell-surface molecules that are required for antigen presentation to T cells and for rapid graft rejection. In humans, the MHC is also known as the human leukocyte antigen or HLA complex. Proteins encoded by the MHC are known as MHC molecules and are classified into class I and class II MHC molecules. Class I MHC molecules are expressed by nearly all nucleated cells and have been shown to function in antigen presentation to CD8⁺ T cells. Class I molecules include HLA-A, B, and C in humans. Class II MHC molecules are known to function in CD4⁺ T cells and, in humans, include HLA-DP, -DQ, and DR.

Antigen presenting cell recruitment factors or APC recruitment factors include both intact, whole cells as well as other molecules that are capable of recruiting antigen presenting cells. Examples of suitable APC recruitment factors include molecules such as interleukin 4 (IL4), granulocyte macrophage colony stimulating factor (GM-CSF), Sepragel and macrophage inflammatory protein 3 alpha (MIP3α). APC recruitment factors also can be produced recombinantly produced. Peptides, proteins and compounds having the same biological activity as the above-noted factors are included within the scope of this invention.

The term immune effector cell refers to cells capable of binding antigens and which mediate an immune response. These cells include, but are not limited to, T cells, B cells, monocytes, macrophages, NK cells and cytotoxic T lymphocytes (CTLs), including CTL lines, CTL clones, and CTLs from tumor, inflammatory, or other infiltrates. Certain diseased tissue expresses specific antigens and CTLs specific for these antigens have been identified.

The term immune effector molecule as used herein, refers to molecules capable of antigen-specific binding, and includes antibodies, T cell antigen receptors, and MHC Class I and Class II molecules.

A naïve immune effector cell is an immune effector cell not bee activated by an antigen. Activation of naïve immune effector cells requires both recognition of the peptide:MHC complex and the simultaneous delivery of a co-stimulatory signal by a professional APC in order to proliferate and differentiate into antigen-specific armed effector T cells.

An educated antigen-specific immune effector cell is an immune effector cell which has previously encountered an antigen. In contrast with its naïve counterpart, activation of an educated antigen-specific immune effector cell does not require a co stimulatory signal. Recognition of the peptide:MHC complex is sufficient.

Immune response broadly refers to the antigen-specific responses of lymphocytes specific for that particular foreign or self antigen. Any substance that can elicit an immune response is said to be immunogenic and is referred to as an immunogen. All immunogens are antigens; however, not all antigens are immunogenic. An immune response of this invention can be humoral (via antibody activity) or cell-mediated (via T cell activation). The immune response may result in the formation of antigen-specific antibodies, the induction of an antigen-specific cellular immune response, the induction of an antigen-specific T cell response. Additionally, other immune responses are antigen-nonspecific. These include innate immune response mediated by Natural killer (NK) cells. The immune response may be directed against proteins associated with conditions, infections, diseases or disorders such as pathogen antigens or antigens associated with cancer cells.

Activated, when used in reference to a T cell, implies that the cell is no longer in G₀ phase of the cell cycle, and begins to produce one or more cytotoxins, cytokines, and other related membrane-associated proteins characteristic of the cell type and is capable of recognizing and binding any target cell that displays the particular antigen on its surface, and releasing its effector molecules.

Inducing an immune response in a subject is well understood in the art and intends that an increase of at least about 2-fold, more preferably at least about 5-fold, more preferably at least about 10-fold, more preferably at least about 100-fold, more preferably at least about 500-fold, and more preferably at least about 1000-fold, or more can be detected or measured in an immune response to an antigen (or epitope) after introducing the antigen (or epitope) into the subject relative to the immune response before introduction of the antigen (or epitope) into the subject. An immune response to an antigen (or epitope), includes, but is not limited to, production of an antigen-specific (or epitope-specific) antibody and production of an immune cell expressing on its surface a molecule that specifically binds to an antigen (or epitope). Methods of determining whether an immune response to a given antigen (or epitope) has been induced are well known in the art. For example, antigen-specific antibody can be detected using any of a variety of immunoassays known in the art, including, but not limited to, ELISA, wherein, for example, binding of an antibody in a sample to an immobilized antigen (or epitope) is detected with a detectably-labeled second antibody (e.g., enzyme-labeled mouse anti-human Ig antibody).

Delivery of nucleic acid expression vectors to suitable immune cells at one or more time points allows for efficient generation of an antibody response. This immune response can immunize an animal against a concurrent or subsequent injection. Antibodies can also be subsequently obtained from the immunized host (e.g., production of polyclonal antibodies by bleeding). Alternatively, monoclonal antibody-producing hybridoma cells can be made by fusing antibody producing B (plasma) cells from the immunized host (e.g., spleen cells) with myeloma cells. Alternatively, the plasma cells can be immortalized, e.g., by retroviral transduction of ABL-Myc. Antibodies can be obtained from immortalized plasma cells (ascites) or hybridoma cells following culture in vitro or in vivo. Alternatively, T cell clones can be generated. Genetic immunization is extremely attractive for those investigators who have difficulty purifying a given protein or synthesizing a peptide. Also, those who already have cDNAs in mammalian expressions vectors can make antibodies quickly.

An antibody is any immunoglobulin, including antibodies and fragments thereof, that binds a specific epitope. The term encompasses polyclonal, monoclonal, and chimeric antibodies. An antibody combining site is that structural portion of an antibody molecule comprised of heavy and light chain variable and hypervariable regions that specifically binds antigen. The phrase antibody molecule in its various grammatical forms as used herein contemplates both an intact immunoglobulin molecule and an immunologically active portion of an immunoglobulin molecule. Exemplary antibody molecules are intact immunoglobulin molecules, substantially intact immunoglobulin molecules and those portions of an immunoglobulin molecule that contains the paratope, including those portions known in the art as Fab, Fab′, F(ab′).sub.2 and F(v), which portions are preferred for use in the therapeutic methods described herein. Fab and F(ab′).sub.2 portions of antibody molecules are prepared by the proteolytic reaction of papain and pepsin, respectively, on substantially intact antibody molecules by methods that are well-known. The phrase monoclonal antibody in its various grammatical forms refers to an antibody having only one species of antibody combining site capable of immunoreacting with a particular antigen. A monoclonal antibody thus typically displays a single binding affinity for any antigen with which it immunoreacts. A monoclonal antibody may therefore contain an antibody molecule having a plurality of antibody combining sites, each immunospecific for a different antigen; e.g., a bispecific (chimeric) monoclonal antibody.

Co-stimulatory molecules are involved in the interaction between receptor ligand pairs expressed on the surface of antigen presenting cells and T cells. Research has demonstrated that resting T cells require at least two signals for induction of cytokine gene expression and proliferation (Schwartz 1990, Jenkins 1992). One signal, which confers specificity, can be produced by interaction of the TCR/CD3 complex with an appropriate MHC/peptide complex. The second signal is not antigen specific and is termed the co-stimulatory signal. This signal was originally defined as an activity provided by bone-marrow-derived accessory cells such as macrophages and dendritic cells, the so called professional APCs. Several molecules have been shown to enhance co-stimulatory activity. These are heat stable antigen (HSA) (Liu et al. 1992), chondroitin sulfate-modified MHC invariant chain (Ii-CS) (Naujokas et al. 1993), intracellular adhesion molecule 1 (ICAM-1) (Van Seventer 1990), B7-1, and B7-2/B70 (Schwartz 1992). These molecules each appear to assist co-stimulation by interacting with their cognate ligands on the T cells. Co-stimulatory molecules mediate co-stimulatory signal(s), which are necessary, under normal physiological conditions, to achieve full activation of naïve T cells. One exemplary receptor-ligand pair is the B7 co-stimulatory molecule on the surface of APCs and its counter-receptor CD28 or CTLA-4 on T cells (Freeman et al. 1993, Young et al. 1992, Nabavi et al. 1992). Other important co-stimulatory molecules are CD40, CD54, CD80, and CD86. The term co-stimulatory molecule encompasses any single molecule or combination of molecules which, when acting together with a peptide/MHC complex bound by a TCR on the surface of a T cell, provides a co-stimulatory affect which achieves activation of the T cell that binds the peptide. The term thus encompasses B7, or other co-stimulatory molecule(s) on an antigen-presenting matrix such as an APC, fragments thereof (alone, complexed with another molecule(s), or as part of a fusion protein) which, together with peptide/MHC complex, binds to a cognate ligand and results in activation of the T cell when the TCR on the surface of the T cell specifically binds the peptide. Co-stimulatory molecules are commercially available from a variety of sources, including, for example, Beckman Coulter, Inc. (Fullerton, Calif.). It is intended that molecules having similar biological activity as wild-type or purified co-stimulatory molecules (e.g., recombinantly produced or muteins thereof) are intended to be used within the spirit and scope of the invention.

The term immunomodulatory agent, as used herein, is a molecule, a macromolecular complex, or a cell that modulates an immune response and encompasses a synthetic antigenic peptide of the invention alone or in any of a variety of formulations described herein; a polypeptide comprising a synthetic antigenic peptide of the invention; a polynucleotide encoding a peptide or polypeptide of the invention; a synthetic antigenic peptide of the invention bound to a Class I or a Class II MHC molecule on an antigen-presenting matrix, including an APC and a synthetic antigen presenting matrix (in the presence or absence of co-stimulatory molecule(s)); a synthetic antigenic peptide of the invention covalently or non-covalently complexed to another molecule(s) or macromolecular structure; and an educated, antigen-specific immune effector cell which is specific for a peptide of the invention.

Modulation of an immune response includes inducing (increasing, eliciting) an immune response; and reducing (suppressing) an immune response. An immunomodulatory method (or protocol) is one that modulates an immune response in a subject.

As used herein, the term cytokine refers to any one of the numerous factors that exert a variety of effects on cells, for example, inducing growth or proliferation. Cytokines, which may be used alone or in combination in the practice of the present invention, may be selected from the group comprising: interleukin-2 (IL-2), stem cell factor (SCF), interleukin3 (IL-3), interleukin6 (IL-6), interleukin 12 (IL-12), G-CSF, granulocyte macrophage-colony stimulating factor (GM-CSF), interleukin-1 alpha (IL-1□), interleukin-11 (IL-11), MIP-11, leukemia inhibitory factor (LIF), c-kit ligand, thrombopoietin (TPO) and Flt3 ligand. It is intended that molecules having similar biological activity as wild-type or purified cytokines (e.g., recombinantly produced or muteins thereof) are intended to be used within the spirit and scope of the invention.

The term peptide is used in its broadest sense to refer to a compound of two or more subunit amino acids, amino acid analogs, or peptidomimetics. The subunits may be linked by peptide bonds. In another embodiment, the subunit may be linked by other bonds, e.g., ester, ether, etc. As used herein the term amino acid refers to either natural and/or unnatural or synthetic amino acids, including glycine and both the D or L optical isomers, and amino acid analogs and peptidomimetics. A peptide of three or more amino acids is commonly called an oligopeptide if the peptide chain is short. If the peptide chain is long, the peptide is commonly called a polypeptide or a protein.

The terms polynucleotide and nucleic acid molecule are used interchangeably to refer to polymeric forms of nucleotides of any length. The polynucleotides may contain deoxyribonucleotides, ribonucleotides, and/or their analogs. Nucleotides may have any three-dimensional structure, and may perform any function, known or unknown. The term polynucleotide includes, for example, single-stranded, double-stranded and triple helical molecules, a gene or gene fragment, exons, introns, mRNA, tRNA, rRNA, ribozymes, cDNA, recombinant polynucleotides, branched polynucleotides, plasmids, vectors, isolated DNA of any sequence, isolated RNA of any sequence, nucleic acid probes, and primers. A nucleic acid molecule may also comprise modified nucleic acid molecules.

A gene delivery vehicle is defined as any molecule that can carry inserted polynucleotides into a host cell. Examples of gene delivery vehicles are liposomes; biocompatible polymers, including natural polymers and synthetic polymers; lipoproteins; polypeptides; polysaccharides; lipopolysaccharides; artificial viral envelopes; metal particles; and bacteria, or viruses, such as baculovirus, adenovirus and retrovirus, bacteriophage, cosmid, plasmid, fungal vectors and other recombination vehicles typically used in the art which have been described for expression in a variety of eukaryotic and prokaryotic hosts, and may be used for gene therapy as well as for simple protein expression. Gene therapy is the purposeful delivery of genetic material to somatic cells for the purpose of treating disease or for biological or medical investigation.

Gene delivery, gene transfer, and the like, as used herein, are terms referring to the introduction of an exogenous polynucleotide (sometimes referred to as a transgene) into a host cell, irrespective of the method used for the introduction. Such methods include a variety of well-known techniques such as vector-mediated gene transfer (by, e.g., viral, infection, transfection, or various other protein-based or lipid based gene delivery complexes) as well as techniques facilitating the delivery of naked polynucleotides (such as electroporation, gene gun delivery, hydrodynamic delivery and various other techniques used for the introduction of polynucleotides). Examples of viral vectors include retroviral vectors, adenovirus vectors, adeno-associated virus vectors, alphavirus vectors and the like. The introduced polynucleotide may be stably or transiently maintained in the host cell. Stable maintenance typically requires that the introduced polynucleotide either contains an origin of replication compatible with the host cell or integrates into a replicon of the host cell such as an extra chromosomal replicon (e.g., a plasmid) or a nuclear or mitochondrial chromosome. A number of vectors are known to be capable of mediating transfer of genes to mammalian cells, as is known in the art and described herein.

In vivo gene delivery, gene transfer, gene therapy and the like as used herein, are terms referring to the introduction of a vector comprising an exogenous polynucleotide directly into the body of an organism, such as a human or non-human mammal, whereby the exogenous polynucleotide is introduced to a cell of such organism in vivo.

A polynucleotide can be delivered to a cell to express an exogenous nucleotide sequence, to inhibit, eliminate, augment, or alter expression of an endogenous nucleotide sequence, or to affect a specific physiological characteristic not naturally associated with the cell. Polynucleotides may contain an expression cassette coded to express a whole or partial protein, or RNA. An expression cassette refers to a natural or recombinantly produced polynucleotide that is capable of expressing a sequence. The term recombinant as used herein refers to a polynucleotide molecule that is comprised of segments of polynucleotide joined together by means of molecular biological techniques. The cassette contains the coding region of the gene of interest along with any other sequences that affect expression of the sequence of interest. An expression cassette typically includes a promoter (allowing transcription initiation), and a transcribed sequence. Optionally, the expression cassette may include, but is not limited to, transcriptional enhancers, non-coding sequences, splicing signals, transcription termination signals, and polyadenylation signals. An RNA expression cassette typically includes a translation initiation codon (allowing translation initiation), and a sequence encoding one or more proteins. Optionally, the expression cassette may include, but is not limited to, translation termination signals, a polyadenosine sequence, internal ribosome entry sites (IRES), and non-coding sequences.

The term gene generally refers to a polynucleotide sequence that comprises coding sequences necessary for the production of a therapeutic polynucleotide (e.g., ribozyme) or a polypeptide or precursor. The polypeptide can be encoded by a full length coding sequence or by any portion of the coding sequence so long as the desired activity or functional properties (e.g., enzymatic activity, ligand binding, signal transduction) of the full-length polypeptide or fragment are retained. The term also encompasses the coding region of a gene and the including sequences located adjacent to the coding region on both the 5′ and 3′ ends for a distance of about 1 kb or more on either end such that the gene corresponds to the length of the full-length mRNA. The sequences that are located 5′ of the coding region and which are present on the mRNA are referred to as 5′ untranslated sequences. The sequences that are located 3′ or downstream of the coding region and which are present on the mRNA are referred to as 3′ untranslated sequences. The term gene encompasses both cDNA and genomic forms of a gene. A genomic form or clone of a gene contains the coding region interrupted with non-coding sequences termed introns, intervening regions or intervening sequences. Introns are segments of a gene, which are transcribed into nuclear RNA. Introns may contain regulatory elements such as enhancers. Introns are removed or spliced out from the nuclear or primary transcript; introns therefore are absent in the mature RNA transcript. The messenger RNA (mRNA) functions during translation to specify the sequence or order of amino acids in a nascent polypeptide. A gene may also include other regions or sequences including, but not limited to, promoters, enhancers, transcription factor binding sites, polyadenylation signals, internal ribosome entry sites, silencers, insulating sequences, matrix attachment regions. These sequences may be present close to the coding region of the gene (within 10,000 nucleotides) or at distant sites (more than 10,000 nucleotides). These non-coding sequences influence the level or rate of transcription and/or translation of the gene. Covalent modification of a gene may influence the rate of transcription (e.g., methylation of genomic DNA), the stability of mRNA (e.g., length of the 3′ polyadenosine tail), rate of translation (e.g., 5′ cap), nucleic acid repair, nuclear transport, and immunogenicity.

Host cell, target cell or recipient cell are intended to include any individual cell or cell culture which can be or have been recipients for vectors or the incorporation of exogenous nucleic acid molecules, polynucleotides and/or proteins. It also is intended to include progeny of a single cell, and the progeny may not necessarily be completely identical (in morphology or in genomic or total DNA complement) to the original parent cell due to natural, accidental, or deliberate mutation. The cells may be prokaryotic or eukaryotic, and include but are not limited to bacterial cells, yeast cells, animal cells, and mammalian cells, e.g., murine, rat, simian or human;

The terms cancer, neoplasm, and tumor, used interchangeably and in either the singular or plural form, refer to cells that have undergone a malignant transformation that makes them pathological to the host organism. Primary cancer cells (that is, cells obtained from near the site of malignant transformation) can be distinguished from non-cancerous cells by well-established techniques, particularly histological examination. The definition of a cancer cell, as used herein, includes not only a primary cancer cell, but also any cell derived from a cancer cell ancestor. This includes metastasized cancer cells, and in vitro cultures and cell lines derived from cancer cells. When referring to a type of cancer that normally manifests as a solid tumor, a clinically detectable tumor is one that is detectable on the basis of tumor mass; e.g., by such procedures as CAT scan, magnetic resonance imaging (MRI), X-ray, ultrasound or palpation. Biochemical or immunologic findings alone may be insufficient to meet this definition.

Suppressing tumor growth indicates a growth state that is curtailed compared to growth without contact with educated, antigen-specific immune effector cells described herein. Tumor cell growth can be assessed by any means known in the art, including, but not limited to, measuring tumor size, determining whether tumor cells are proliferating using a 3H-thymidine incorporation assay, or counting tumor cells. Suppressing tumor cell growth means any or all of the following states: slowing, delaying, and suppressing tumor growth indicates a growth state that is curtailed when stopping tumor growth, as well as tumor shrinkage.

A pharmaceutical composition is intended to include the combination of an active agent with a carrier, inert or active, making the composition suitable for diagnostic or therapeutic use in vitro, in vivo or ex vivo.

As used herein, the term pharmaceutically acceptable carrier encompasses any of the standard pharmaceutical carriers, such as a phosphate buffered saline solution, water, and emulsions, such as an oil/water or water/oil emulsion, and various types of wetting agents. The compositions also can include stabilizers and preservatives. For examples of carriers, stabilizers and adjuvants, see Martin REMINGTON'S PHARM. SCL, 15th Ed. (Mack Publ. Co., Easton (1975)).

An effective amount is an amount sufficient to effect beneficial or desired results. An effective amount can be administered in one or more administrations, applications or dosages.

The practice of the present invention employs, unless otherwise indicated, conventional techniques of molecular biology (including recombinant techniques), microbiology, cell biology, biochemistry and immunology, which are within the skill of the art. Such techniques are explained fully in the literature. See e.g., Sambrook et al., Molecular Cloning: A Laboratory Manual, 2.sup.nd edition (1989); Current Protocols in Molecular Biology (F. M. Ausubel et al., eds (1987)); the series Methods in Enzymology (Academic Press, Inc.); Antibodies, A Laboratory Manual (Harlow and Lane eds. (1988)); and Animal Cell Culture (R. I. Freshney ed. (1987)).

A therapeutic effect of an expressed protein in attenuating or preventing a disease state can be accomplished by the protein either staying within the cell, remaining attached to the cell in the membrane or being secreted and dissociating from the cell where it can enter the general circulation and blood. Secreted proteins that can be therapeutic include hormones, cytokines, growth factors, clotting factors, anti-protease proteins (e.g., alpha-antitrypsin), and other proteins that are present in the blood. Proteins on the membrane can have a therapeutic effect by providing a receptor for the cell to take up a protein or lipoprotein. For example, the low density lipoprotein (LDL) receptor could be expressed in hepatocytes and lower blood cholesterol levels, thereby preventing the formation of atherosclerotic lesions that can cause strokes or myocardial infarction. Therapeutic proteins that stay within the cell can be enzymes that clear a circulating toxic metabolite as in phenylketonuria. They can also cause a cancer cell to be less proliferative or cancerous (e.g. less metastatic). A protein within a cell could also interfere with the replication of a virus.

### EXAMPLES

The following examples are provided in order to demonstrate and further illustrate certain preferred embodiments and aspects of the present invention and are not to be construed as limiting the scope thereof.

### Example 1

### Hydrodynamic Tail Vein (HTV) Delivery of Plasmid DNA into Mice

HTV injection of the pDNA was performed as described (U.S. Pat. No. 6,627,616 which is incorporated herein by reference). Briefly, pDNA was diluted in pharmaceutically acceptable carrier-solution and injected in a volume of 1 ml per 10 g animal weight during a relatively short time span via tail-vein.

### Example 2

### Hydrodynamic Limb Vein (HLV) Delivery of Plasmid DNA into Mice

HLV injection of pDNA was performed as described in U.S.-2004-0242528, which is incorporated herein by reference. As an example, the pDNA solution was HLV delivered in mice by injection into a distal site in the great saphenous vein of the mouse hind limb. The pDNA was administered in 1.0 ml of normal saline solution (NSS) at a rate of 8.0 ml/min. Just prior to injection, blood flow to and from the limb was restricted by placing a tourniquet around the upper leg just proximal to, or partially over, the quadriceps muscle group. The tourniquet remained in place during the injection and for 2 min post-injection.

### Example 3

### Plasmid DNA Expression Vectors

To administer factors that influence the development, expansion and maturation status of DCs in vivo, pDNA cassettes that express proteins or protein fragments able to directly or indirectly modulate DCs were delivered by intravascular hydrodynamic (HTV or HLV) pDNA delivery. DC modulating factors include molecules that provide signaling to DCs directly through receptor-ligand interactions. As an example, CD40-Ligand (CD40-L, a DC modulating factor) interacts with cell surface expressed CD40 on some DCs, and result in cell signaling events that promote further DC maturation. Other modulating factors may act on precursor cells to increase DC development. As example, Flt3-L (DC modulating factor) acts on CD34⁻ progenitor cells, such as those found in the bone-marrow, to stimulate DC development above the steady state level. Still other DC modulators may act indirectly via a cascading pathway. As example, IL2 (DC modulator) activates NK cells that produce IFNγ and, in concert with NK/DC cell-to-cell contact, promote DC maturation (Gerosa F et. al., J. Immunol. 174: 727 2005).

Several plasmid DNA expression vectors were employed in the following studies and examples. Each of the following vectors used the human cytomegalovirus (CMV) immediate-early promoter to drive expression: pMIR0048 expressed a form of firefly luciferase; pMIR0274, human gp100; pMIR0275, murine gp100; pMIR0532, murine Flex (pUMVC3-mFlex, Aldevron, Fargo, N. Dak.); pMIR0544, murine GM-CSF (pORF9-mGM-CSF, Invivogen, San Diego, Calif.); and pMIR0502, multimeric murine CD40-L (pSP-D-CD154, Haswell et al. 2001). pMIR0261 expresses murine Flt3-L under transcriptional control of the EF-1 promoter (pORF-mFlt3L, Invivogen). All plasmid DNAs were produced endotoxin free by Aldevron. The CMV promoter is known to provide sustained expression when delivered to muscle cells.

### Example 4

### Sequential Gene Delivery of mFlt3-L Plus mGM-CSF pDNA Increases CD83⁺/CD86⁺ Cells in the Spleen

Using the HTV method, 10 μg of murine Flt3-L-expressing pDNA (pORF-mFlt3L) was delivered to ICR mice (obtained from Harlan). This was followed 10 days later by a subsequent HTV delivery of 2.5 μg of murine GM-CSF-expressing pDNA (pORF9-mGM-CSF). The mice were sacrificed and spleens collected for DC analysis four days following GM-CSF delivery (study day 14). Splenocytes were processed to individualize cells, deplete erythrocytes by hypotonic shock, counted, and stained with antibodies (Abs) and assessed for CD11c⁺/CD83⁺/CD86⁺ (i.e., a phenotypically-defined mature DC) and CD11b⁺/Ly-6G⁺ (myeloid suppressor cell) phenotypes by flow cytometry. The results, shown in Table 1, indicate the effects of single-agent, concurrent and sequential combinatorial gene delivery. Groups (n=3) of ICR mice received were injected as follows: (1) no treatment, (2) 10 μg pORF-mFlt3L on day 0, (3) 2.5 μg pORF9-mGMCSF on day 10, (4) 10 μg pORF-mFlt3L on day 0+2.5 μg pORF9-mGMCSF on day 10, (5) 2.5 μg pORF9-mGMCSF on day 0, and (6) 10 μg pORF-mFlt3L+2.5 μg pORF9-mGMCSF on day 0. Isolated splenocytes were 3-color stained with primary-conjugated mAbs to murine CD11c (CD11c-APC; BD Biosciences, San Jose, Calif.), CD86 (CD86-FITC; BD Biosciences), and CD83 (CD83-PE; eBiosciences, San Diego, Calif.) or 2-color stained with primary-conjugated mAbs to murine CD11b (CD11b-FITC; BD Biosciences) and Ly-6G (Ly-6G-PE; BD Biosciences).

TABLE 1
Modulation in Splenic DC Profile

| | | ²percent | ³percent | ⁴percent | ⁵number |
|---|---|---|---|---|---|
| | ¹Cells/ | CD11c⁺ | CD86⁺/ | CD11b⁺/ | mature |
| Treatment (day) | spleen | cells | CD83⁺ | Ly-6G⁺ | DCs |
| No Treatment | 90 | 1.5 | 2.4 | 3.8 | 0.03 |
| mFlt3-L (d 0), 10 μg | 287 | 8.1 | 1.0 | 8.9 | 0.23 |
| mGM-CSF (d 10), | 400 | 11.1 | 36.4 | 34.7 | 16.16 |
| 2.5 μg | | | | | |
| mFlt3-L (d 0) + | 850 | 34.1 | 18.8 | 13.3 | 54.49 |
| mGM-CSF (d 10) | | | | | |
| mGM-CSF (d 0) | 400 | 8.6 | 46.8 | 18.9 | 16.10 |
| mFlt3-L (d 0) + | 250 | 15.0 | 25.4 | 8.4 | 9.52 |
| mGM-CSF (d 0) | | | | | |

¹Number of cells/spleen. Values are ×10⁶ cells/spleen.
²Value represents the percent of viable splenocytes positive for CD11c staining.
³Value represents percent viable CD11c⁺ cells also positive for CD86 and CD83.
⁴Value represents percent viable splenocytes positive for CD11b and Ly-6G.
⁵Mature CD86⁺/CD83⁺ DCs determined by the formula: # of mature DCs = viable cells/spleen × % CD11c⁺ cells × % CD86⁺/CD83⁺ cells. Values are ×10⁶ cells/spleen.

In this experiment, spleens from non-treated control mice contained an average of 90×10⁶ cells, of which 1.5% were CD11c⁺. Of these CD11c⁺ cells, only 2.4% exhibited a mature DC phenotype as determined by CD83⁺/CD86⁺ cells, resulting in only 0.03×10⁶ mature DCs/spleen (0.04%). In contrast, spleens from mice that received sequential Flt3-L/GM-CSF HTV delivery contained an average of 850×10⁶ cells, of which 34.1% were CD11c⁺. Of these CD11c⁺ cells, 18.8% exhibited a mature DC phenotype (CD83⁺/CD86⁺), resulting in greater than 54×10⁶ mature DCs/spleen (6.41%). This gene delivery process resulted in a greater than 1800-fold increase in the number of splenic CD11c/CD83⁺/CD86⁺ cells. Although HTV delivery of Flt3-L alone resulted in increased spleen size (287×10⁶ cells/spleen) and elevated level of CD11c⁺ cells (8.1%), very few of these CD11c⁺ cells exhibited a mature DC phenotype (1.0%), and coincided with the well known fact that Flt3-L treatment alone preferentially expands immature DCs. Concurrent co-delivery of both factors resulted in a similar increase in spleen size (250×10⁶ cells/spleen) as with Flt3-L treatment alone, but a greater percent of splenocytes were CD11c⁺ (15.0%), with 25.4% of these exhibiting a mature DC phenotype. While concurrent Flt3-L/GM-CSF gene delivery resulted in an expansion of CD11c⁺/CD83⁺/CD86⁺ splenocytes (9.5×10⁶ cells/spleen), sequential delivery was significantly superior (5.7-fold greater number of CD11c⁺/CD83⁺/CD86⁺ cells/spleen).

These data demonstrate that sequential gene delivery of pDNA expressing Flt3-L followed by GM-CSF resulted in the largest number of splenic CD11c⁺/CD83⁺/CD86⁺ mature DCs. Sequential delivery of these DC-modulation factors resulted in a greater absolute number of CD11c⁺/CD83⁺/CD86⁺ mature DCs than achieved by concurrent delivery of these factors, or that achieved by single-factor delivery. It is reasonable to expect that similar expansion and maturation of DCs would be achieved if these DC-modulating factors are administered sequentially as protein or biologically active protein fragments. The gene delivery approach permits continuous delivery of these factors over at least several days following a single gene delivery procedure. Data with reporter genes has shown expression of the encoded protein over several days. Therefore, administration of multiple doses of DC-modulating factors in protein form may provide optimum results.

### Example 5

### Sequential Gene Delivery of mFlt3-L Plus mGM-CSF pDNA Increases CD83⁺/CD86⁺ Cells in Axillary Lymph Nodes

We evaluated HTV delivery of Flt3-L (10 μg pORF-mFlt3L) in combination with a lower dose of pORF9-mGM-CSF (2.0 μg). Axillary lymph nodes (ALN) from these mice were evaluated by flow cytometry in a similar fashion as for Example 4. The results, shown in Table 2, indicate that sequential Flt3-L/GM-CSF treatment represents the most effective combination of gene delivery tested (>35-fold increase in mature DCs as compared to controls) to promote expansion and maturation of DCs in lymphoid compartments in addition to spleen. In addition, these data show that the HTV procedure can facilitate DC modulation systemically within the recipient animal.

TABLE 2
Auxiliary Lymph Node DC Profile

| | percent | ¹percent | |
|---|---|---|---|
| | CD11c⁺ | CD86⁺/ | % mature |
| Treatment | cells | CD83⁺ | DCS² |
| No Treatment | 0.2 | 1.4 | 0.003 |
| mFlt3-L (d 0), 10 μg | 1.2 | 4.7 | 0.056 |
| mGM-CSF (d 10), 2.0 μg | 0.4 | u.d. | u.d. |
| mFlt3-L (d 0) + mGM-CSF (d 10) | 1.4 | 7.6 | 0.106 |
| mGM-CSF (d 0) | 0.1 | 3.9 | 0.004 |
| mFlt3-L (d 0) + mGM-CSF (d 0) | 1.3 | 3.1 | 0.040 |

¹Value represents percent viable CD11c⁺ cells also positive for CD86 and CD83.
²% mature DCs = % CD11c⁺ cells × % CD86⁺/CD83⁺ cells from the CD11c⁺ gated cells.
u.d. = not determined

### Example 6

### Gene Delivery of mGM-CSF pDNA

HTV delivery of GM-CSF alone demonstrated a significant impact on splenic DC generation. HTV delivery of 2.5 μg pORF9-mGM-CSF increased spleen size to 400×10⁶ cells/spleen on day 4 or 14 following gene delivery (Table 1). This increase is greater than that achieved by Flt3-L single-agent or concurrent Flt3-L/GM-CSF co-delivery. Although the frequency of CD11c⁺ cells was similar in between Flt3-L or GM-CSF (d0 or d10 delivery) single-agent delivery, GM-CSF alone gave a significantly greater percentage of these cells exhibited the mature DC phenotype (36.4 and 46.8% CD83⁺/CD86⁺ for d0 and d10 pDNA delivery, respectively) resulting in 16.1×10⁶ CD11c⁺/CD83⁺/CD86⁺ per spleen. GM-CSF single-agent delivery resulted in the greatest increase in myeloid suppressor (CD11b⁺/Ly-6G⁺) cells (34.7% [d10 delivery] and 18.9% [d0 delivery] of total splenocytes) as compared to all other treatment groups. Thus, while administration of GM-CSF alone can promote DC expansion and maturation, this method has a greater risk of suppressing immunity as a result of concomitantly increasing the number of myeloid suppressor cells which can act to inhibit and prevent immune function. If the goal is to generate high numbers of myeloid suppressor cells for in vivo, ex vivo or in vitro research, single-factor delivery of GM-CSF would be well suited as a method to promote such expansion.

### Example 7

### Splenocytes, Containing a High Percentage of CD83⁺/CD86⁺ Mature DCs, Produce IL-12 Following CD40 Ligation

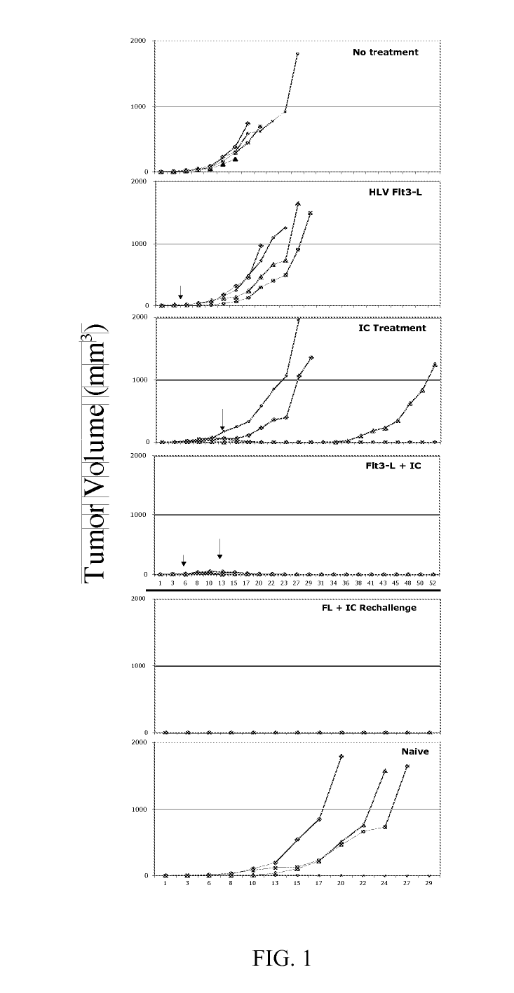

Ligation of the CD40 molecule expressed on the cell surface of murine DCs results in triggering IL12 production by DCs that can be detected in culture supernatants by mIL12 ELISA (Koch F et al. J Exp Med, 184: 741-746, 1996.). The flow cytometric analysis in Table 1 indicated that sequential gene delivery of Flt3-L/GM-CSF resulted in a large expansion of CD11c⁺/CD83⁺/CD86⁺ splenocytes. To functionally demonstrate that these cells are indeed DCs, we tested their activation by CD40-cross-linking and determined whether IL12 was produced. Groups (n=3) of mice received HTV gene delivery as follows: (Control) no treatment, (Flt3-L) 10 μg pORF-mFlt3L on d0, (GM-CSF) 2.0 μg pORF9-mGMCSF on d0, (Concurrent) 10 μg pORF-mFlt3L+2.0 μg pORF9-mGMCSF on d0, and (Sequential) 10 μg pORF-mFlt3L on d0+2.0 μg pORF9-mGMCSF on d10. Spleens were harvested on d14 and individualized, erythrocyte depleted, counted, and profiled by flow cytometry. Group-pooled splenocytes (4×10⁶/ml) were co-cultured with agonistic anti-CD40 Ab (clone FGK 45.4; Buhtoiarov I N et al. J Immunol, 174: 6013-6022, 2005) or control rat IgG Ab in 1 ml of complete medium in a 24-well plate at 37° C. in 5% CO₂ and analyzed for IL12 production by murine IL12p40 ELISA (R&D Systems). As shown in FIG. 1, splenocytes from non-treated mice demonstrated no modulation in IL12 production following CD40-ligation (0.58 and 0.26 ng/ml of IL12 following co-culture with rat IgG or anti-CD40 mAb, respectively). In comparison, splenocytes from mice treated by sequential Flt3-L/GM-CSF were activated and produced IL12 (2.94 ng/ml) in the absence of any additional perturbation (i.e., rat IgG co-culture). CD40-ligation resulted in additional stimulation and increased the IL12 production level to 11.54 ng/ml. Splenocytes from mice treated with concurrent co-delivery of Flt3-L/GM-CSF exhibited a lesser CD40-ligation-induced increase in IL12 production (rat IgG, 2.01 ng/ml; anti-CD40 mAb, 5.55 ng/ml), as might be predicted based on the results presented in Table 1.

### Example 8

### Sequential Flt3-L Plus GM-CSF Gene Delivery Boosts Humoral Response Following Genetic Immunization

To test whether sequential Flt3-L/GM-CSF delivery enhances Ag-specific immune activation, mice were pretreated by sequential HTV (10 μg pORF-mFlt3L d0, 2 μg pORF9-mGM-CSF d10) or HLV (150 μg pORF-mFlt3L d0, 50 μg pORF9-mGM-CSF d10) gene delivery. Mice were then genetically immunization by HLV delivery in both limbs with 50 μg of pDNA coding for luciferase or the melanoma tumor Ag human gp100 (hgp100) on d10. Group-pooled (n=3) sera obtained on d20 and d34 were evaluated for anti-luciferase Abs by ELISA. A standard curve was developed using a commercially available primary mouse-anti-luciferase monoclonal antibody. Data reported as mean values (μg/ml) for group-pooled sera collected on a specific date.

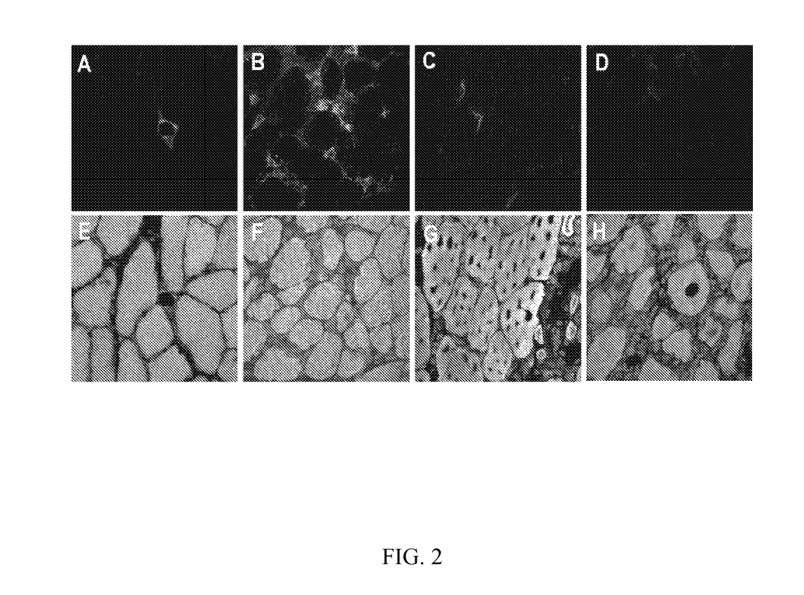

The data in FIG. 2 indicate that combining genetic immunization with the sequential Flt3-L/GM-CSF gene therapy by HLV delivery resulted in the earliest and highest anti-luciferase Ab titers measured. As anticipated, all regimens that included genetic immunization with hgp100 DNA (pCI-hgp100) exhibited no detectible anti-luciferase Ab titers (values <0.36 μg/ml) in the sera at any time-point. Sera from mice that received sequential HLV delivery of Flt3-L/GM-CSF in combination with luciferase genetic immunization exhibited anti-luciferase titers of 22.71 μg/ml at the earliest time-point tested, d20 (corresponding to d10 post-immunization). These titers were >8.5-fold higher than titers observed from mice that were immunized but did not receive sequential Flt3-L/GM-CSF gene delivery (2.67 μg/ml). An enhanced humoral response continued to be maintained. Day 34 samples indicated a titer of 245.39 μg/ml in combined HLV sequential Flt3-L/GM-CSF plus genetic immunized mice, and a lower titer of 136.86 μg/ml in the immunized only mice. The data in FIG. 2 demonstrate that sequential Flt3-L/GM-CSF gene delivery, which increases the number of DCs in vivo, can enhance immune reactivity (observed as increased Ab titers) when administered under appropriate conditions.

### Example 9

### The Sequential Delivery of Flt3-L and GM-CSF Promotes Influx of Mature DCs

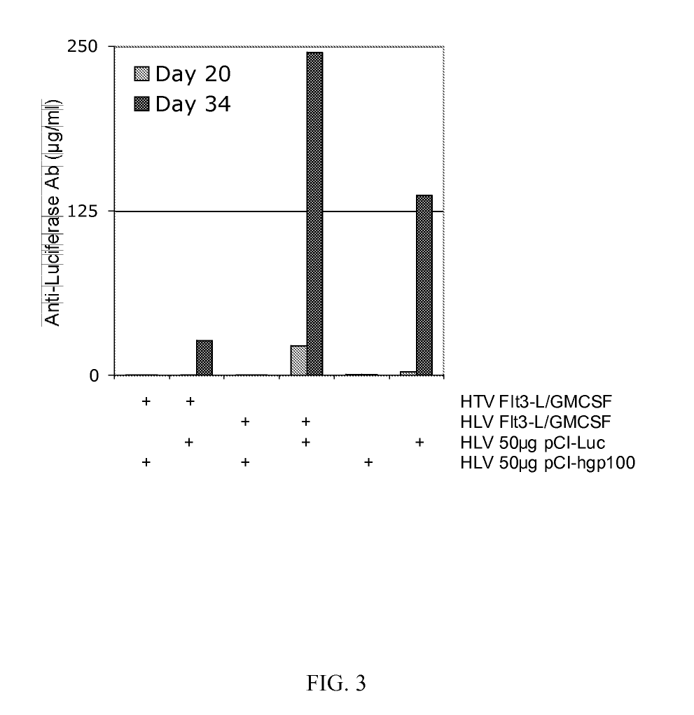

The results in Example 8 indicate that sequential HLV delivery of Flt3-L/GM-CSF enhances humoral immunity, and is the result of an increased presence of mature DCs in the treated limb during the HLV genetic vaccination procedure. The relative presence of DCs was assessed by immunohistochemistry in the hind limbs that received 200 μg pORF-mFlt3L d0 followed by 50 μg pORF9-mGM-CSF d10. Muscle tissue was harvested 4 d after GM-CSF delivery. The Flt3-L/GM-CSF-treated limb was compared to the untreated contralateral limb. Histological analysis of muscle tissues from untreated limbs showed normal morphology with no infiltration of DCs (FIGS. 3A and 3E). This observation is consistent with previous reports on the near-absence of DCs in skeletal muscle (Pimorady-Esfahani et al. Muscle Nerve 20:158-166, 1997.). Muscle tissue harvested from limbs transfected with mFlt3-L and mGM-CSF pDNAs showed a large influx of mononuclear cells between muscle fibers (FIG. 3B-H). It has previously been shown that the hydrodynamic limb vein gene delivery procedure is not itself associated with inflammatory infiltration, even after repeated procedure (Toumi and Hagstrom Mol. Ther. 13:229-36 2006). Most of the mononuclear cellular infiltrates could be labeled with an anti-CD11c antibody, a common DC marker (FIGS. 3B and 3F). The number of infiltrating CD11c⁺ cells is much greater than that observed by others after direct intramuscular GM-CSF pDNA delivery (Haddad D et al. J Immunol 165:3772-3781, 2000). Many infiltrating cells were positive for CD83 staining, indicating recruitment of mature DCs (FIGS. 3C and 3G). Immunostaining with isotype-matching Abs showed no or very little background reactivity (FIGS. 3D and 3H), demonstrating specificity of the anti-CD11c and anti-CD83 antibody staining.

These results show that sequential delivery of Flt3-L/GM-CSF to muscle promotes the influx of mature DCs into the muscle tissue. When combined with immunization or vaccine procedures that utilize the same tissue compartment for Ag expression or site of Ag administration, greater immunity is potentiated. For enhanced vaccine-induced immunity, the invention-expanded pool of DCs must be available to acquire the vaccine-provided antigen. If DC-expansion occurs in a physiological compartment that does not get exposed to vaccine-provided antigen, augmented processing and presentation of Ag to T cells will not be enhanced.

### Example 10

### Sequential Flt3-L/GM-CSF Treatment Increases the Frequency of Ag-Specific T Cells following vaccination

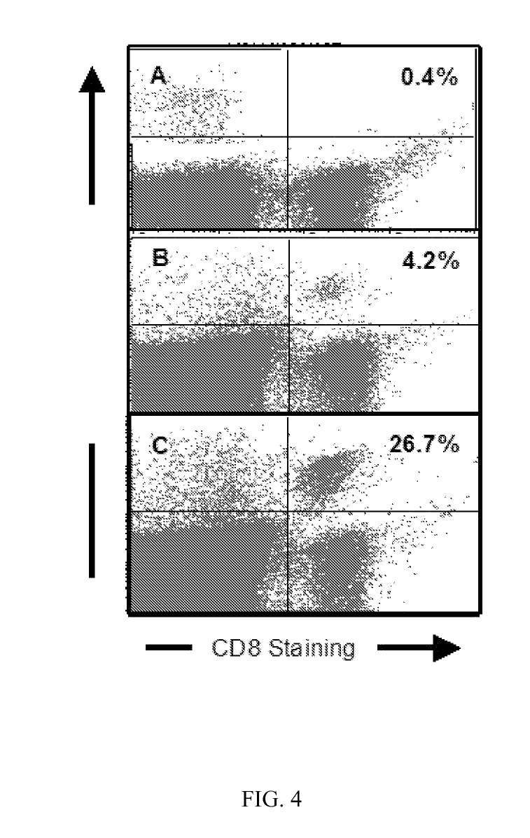

Examples 8 and 9 show that sequential Flt3-L/GM-CSF administration results in mature DC infiltration that can augment humoral immunity. When Flt3-L/GM-CSF administration is combined with vaccination, the frequency of Ag-specific T cells can also be increased. Groups (n=3) of mice received a single HLV delivery of 50 μg of hgp100 pDNA in both the right and left hind-limbs on d10 (FIG. 4B) or 200 μg Flt3-L pDNA on d0 and 50 μg of GM-CSF+50 μg of hgp100 pDNA (mixed) in both right and left hind-limbs on d10 (FIG. 4C). Negative control sample is from naïve mice (FIG. 4A). Peripheral blood mononuclear cells (PBMCs) were assessed for the presence of double positive cells staining for murine CD8 and the hgp10025-33/H2 Db-tetramer. Cells were 2-color stained for murine CD8-FITC and hgp10025-33/H2 Db-tetramer-PE and analyzed by flow cytometry on d14. Tetramer analysis was used to identify Ag-specific T cells from blood of treated animals. The Ag tested was the human gp100 molecule (hgp100), the human homologue of the murine gp100 (mgp100) melanoma tumor associated antigen (TAA), which is able to break immune tolerance against the mgp100 and induce protection against B16 murine melanoma tumor challenge (Gold J S et al. J Immunol 170:5188-5194, 2003). For these particular analyses, tetramers were comprised of the hgp100₂₅₋₃₃ peptide (an immunodominant epitope in C57BL/6 strain mice) bound to the H2D^{b}-MHC class 1 molecule, complexed together in a multimeric form. We determined the percentage of hgp100₂₅₋₃₃/H2D^{b}-tetramer⁺CD8⁺ T cells in the peripheral blood 4 days after vaccination of C57BL/6 mice. As shown in FIG. 4B, a single HLV delivery of 50 μg of hgp100 pDNA into both hind limbs resulted in 4.2% of all CD8⁺ T cells being specific for the gp100₂₅₋₃₃ Ag epitope. When combined with sequential Flt3-L/GM-CSF gene delivery, the single HLV delivered hgp100 vaccine resulted in nearly 27% of CD8⁺ T cells stained with the gp100₂₅₋₃₃/H2D^{b} tetramer (FIG. 4C). This combinatorial approach facilitated a greater than 6-fold increase in the percentage of Ag-specific CD8⁺ T cells. A cancer vaccine strategy that can elevate the number of Ag-specific CD8⁺ T may have great therapeutic potential in the treatment of cancer.

### Example 11

### Sequential Flt3-L/GM-CSF Treatment Enhances Vaccine-Induced Antitumor Immunity

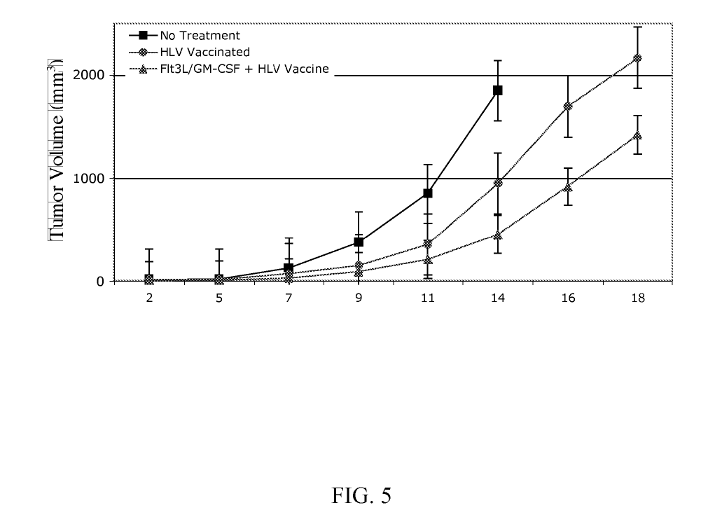

The increase in frequency of Ag-specific T cells as a result of combining sequential Flt3-L/GM-CSF with vaccination (Example 10) translates into greater antitumor immunity. Groups (n=4) of naïve mice were genetically vaccinated by HLV delivery of 50 μg of hgp100 pDNA in the right hind limb on d10 (circle, FIG. 5) or combinatorial vaccinated (triangle, FIG. 5) by HLV delivery of 200 μg of Flt3-L pDNA on d0 followed by 50 μg of GM-CSF+50 μg of hgp100 pDNA (mixed and concurrently injected) on d10. Following vaccination, mice were challenged on day 20 with a high-dose (1×10⁶ cells) of B16 tumor cells. Naïve control mice were also challenged (square, FIG. 5). FIG. 5 shows tumor growth in mice challenged with the 1×10⁶ B16 tumor cell dose. Mice that were HLV hgp100 vaccinated exhibited an antitumor response observed as depressed tumor growth, with statistical differences in tumor burden between HLV vaccinated and naïve mice on all days subsequent to d5 post-tumor challenge (d14: p=0.00589). Likewise, there was an evident antitumor response in mice that received the combination of sequential Flt3-L/GM-CSF plus hgp100 vaccine when compared to tumor growth in naïve mice (d14: p<0.00001). Most importantly, there was a greater antitumor effect realized in mice that received the Flt3-L/GM-CSF plus vaccine compared with vaccine alone. A significant difference in tumor growth was noted between these two vaccination groups beginning on d14 following tumor challenge (d14: p=0.01684).

Representative mice were bled and assessed for percent of antigen-specific CD8⁺ T cells by hgp100₂₅₋₃₃/H2 Db tetramer staining of PBMCs just prior to tumor inoculation. HLV vaccinated mice exhibited 3.2% tetramer positive staining CD8⁺ T cells, while combinatorial vaccinated mice showed 17.2% tetramer positive CD8⁺ T cells; control mice exhibited 0.6% tetramer staining CD8⁺ T cells. These data concur with that in Example 10, indicating that a strategy that includes sequential Flt3-L/GM-CSF treatment in conjunction with vaccination, results in an increased frequency of Ag-specific T cells that can mediate greater immunity against tumor challenge. These results, obtained using a well-known melanoma TAA, indicate that similar strategies to enhance an immune response, such as vaccination against infectious diseases, will also be analogously enhanced.

### Example 12

### Systemic Production of Biologically Active Flt3-L by Gene Delivery

DC-modulating factors can be administered in a variety of forms, such as proteins, protein fragments, peptides, or expressed molecules following delivery of pDNA expression cassettes. Gene delivery, such as by HTV or HLV delivery, of pDNA expressing full-length mFlt3-L (pORF-mFlt3L) or extracellular secreted form of mFlt3-L (mFLex; pUMVC3-mFLex) results in expression of systemically available mFlt3-L molecules.

HTV delivery of 10 μg of pORF-mFlt3L or pUMVC3-mFLex resulted in serum levels of 11812 ng/ml and 27375 ng/ml of mFlt3-L protein, respectively, in the serum 24 hr following gene delivery as determined by ELISA (R&D Systems, Minneapolis, Minn.). Group-pooled (n=3) sera from ICR strain mice was collected 24 hr following HTV gene delivery and assessed for the amount of mFlt3-L present in the sera by ELISA according to the manufacturer's instructions. This amount of Flt3-L protein in the sera is similar to that reported by others evaluating HTV delivery of human Flex pDNA (He Y et al. Hum Gene Ther, 11: 547-554, 2000).

HLV delivery of Flt3-L (in combination with sequential GM-CSF delivery) promoted a local influx of DCs into the muscle tissue (see Example 9, FIG. 3) that promoted increased T cell mediated antitumor immunity when combined with a HLV delivered cancer vaccine (Examples 10 and 11). In addition, HLV delivery of Flt3-L results in systemically available Flt3-L molecules that can modulate the cellular profile of distally located tissue compartments. The results in Table 3 indicate the mFlt3-L serum values and phenotypic profile of splenocytes following HLV delivery of mFlt3-L or mFLex. Group-pooled serum was collected 72 hr following HLV gene delivery and evaluated by ELISA to quantitate serum mFlt3-L levels. HLV delivery of 200 μg of pORF-mFlt3L to both hind limbs resulted in a serum level of 1.70 ng/ml of mFlt3-L 72 hr post-delivery. Spleens harvested at d9 following injection showed a 2.5-fold increase in cell number as compared to non-treatment controls (70×10⁶ cell/spleen and 26×10⁶ cell/spleen, respectively), and resulted in a 4.6-fold increase in the number of CD11c⁺ splenic DCs and 3.6-fold elevation in the number of splenic NK cells. HLV delivery of 200 μg of pUMVC3-mFLex resulted in 10.35 ng/ml of mFlt3-L at 72 hr post-delivery, and represents a 5-6-fold increase in mFlt3-L systemically available as compared to pORF-mFlt3L (1.7 ng/ml) delivery. This higher level of mFlt3-L serum levels correlated with even greater increases in splenic DCs and NK cell numbers, with an 11-fold and 4.2-fold increase, respectively, as compared to control animal values.

TABLE 3
HLV Delivery of mFlt3-L

| | Flt3-L | Cells/ | Splenic phenotypic profile⁴ | | | | Total | Total |
|---|---|---|---|---|---|---|---|---|
| Treatment¹ | Level² | spleen³ | CD4⁺ | CD8⁺ | CD11c⁺ | DX5⁺ | DCs⁵ | NKs⁶ |
| No Treatment | 0.1 | 28 | 12.9 | 8.7 | 1.1 | 3.5 | 0.3 | 1.0 |
| mFLex (RT limb) | 4.84 | 64 | 12.3 | 9.0 | 3.3 | 5.2 | 2.1 | 3.3 |
| mFlt3-L (RT limb) | 0.87 | 56 | 13.8 | 9.7 | 1.7 | 4.8 | 0.9 | 2.7 |
| mFLex (Both) | 10.35 | 76 | 12.2 | 9.3 | 4.3 | 5.5 | 3.3 | 4.2 |
| mFlt3-L (Both) | 1.70 | 70 | 12.4 | 9.4 | 2.0 | 5.1 | 1.4 | 3.6 |
| mFLex (Sequential) | 5.06, | 80 | 11.5 | 9.4 | 3.4 | 4.8 | 2.7 | 3.8 |
| | 5.65 | | | | | | | |
| mFlt3-L (Sequential) | 1.69, | 66 | 12.4 | 8.6 | 1.6 | 4.4 | 1.1 | 2.9 |
| | 1.01 | | | | | | | |
| ¹Groups (n = 4) of A/J strain mice received gene delivery as follows: (1) no treatment, (2) hydrodynamic limb vein injection (HLV) of 200 μg murine Flt3-L extracellular secreted (mFLex) DNA to right limb on d 0, (3) HLV of 200 μg mFlt3-L DNA to right limb d 0, | | | | | | | | |
| # (4) HLV of 200 μg mFLex DNA to both limbs on d 0, (5) HLV of 200 μg mFlt3-L DNA to both limbs on d 0, (6) HLV of 200 μg mFLex DNA to right limb on d 0 plus 200 μg mFLex DNA to left limb on d 6, and (7) HLV of 200 μg mFlt3-L DNA to right limb on d 0 plus 200 μg mFlt3-L DNA to left limb on d 6. | | | | | | | | |

²Serum murine Flt3-L concentration (ng/ml) of pooled sera 72 hr following HLV gene delivery as determined by ELISA (R&D Systems, Minneapolis, MN). Additional values for Groups 6 and 7 represents serum levels 72 hr following second HLV injection.
³Spleens were harvested on d 9 and pooled for each group. Following erythrocyte lysis by hypotonic shock, the number of viable cells was determined. Values are ×106 cells/spleen.
⁴Group-pooled isolated splenocytes were stained with primary-conjugated mAbs (BD Biosciences, San Diego, CA) to murine CD4, CD8, CD11c, and DX5. Value represents the percent of viable splenocytes positive for specific cell-surface staining.
⁵Indicates the total number of CD11c⁺ DCs per spleen and is determined by the formula: = number of viable cells/spleen × % CD11c⁺ cells. Values are ×106 cells/spleen.
⁶Indicates the total number of DX5⁺ NKs per spleen and is determined by the formula: = number of viable cells/spleen × % DX5⁺ cells. Values are ×106 cells/spleen.

These results demonstrate that limb muscle delivery and expression of Flt3-L is capable of increasing the number and modulating the population of splenic immune cellular constituents. As already shown in Examples 8, 10 and 11, HLV delivery of Flt3-L can be important in efforts to enhance humoral and cellular immunity. Given the clinical applicability of HLV gene delivery, the ability to also increase the number of splenic NK effectors would have an import impact in strategies that seek to augment NK-mediated immune responses. As example, the hu14.18-IL2 immunocytokine mediates tumor destruction by an NK-dependent mechanism (Neal Z C et al. Cancer Immunol Immunother, 53: 41-52, 2004). Therefore, combining HLV Flt3-L treatment with immunocytokine therapy is expected to increase antitumor efficacy, in part, by expanding the pool of potential NK effectors in vivo.

### Example 13

### DC Isolation and Cyopreservation

For application as a source of primary mature DCs for in vitro and ex vivo manipulation, mature DCs can be isolated from other splenocytes and cryopreserved for future use and distribution.

Following Flt3-L/GM-CSF administration, significant expansion and maturation of DCs has occurred in lymphatic compartments such as the spleen. To obtain a near-pure mature DC population from spleen, a simple purification protocol is performed. The first step is the separation of monocytes from lymphocytes on a high-density hyper-osmotic Percoll density gradient as described by others (Repnik U et al. J Immunol Methods. 278:283-92 2003). Isolation of the monocyte-containing layer results in a 2-3-fold enrichment with 65-70% of recovered cells being CD11c⁺ DCs. Next, CD83⁺ mature DCs are selected for using a positive-selection process such as magnetic separation column (Miltenyi Biotec, Auburn, Calif.). For murine DC application, anti-murine CD83 mAb (clone Michel-17) is used which recognizes and binds CD83 without directly activating murine DCs (Wolenski M et al. Scand J Immunol, 58: 306-311, 2003). Following the CD83-selection procedure, the effluent is comprised of >90% CD11c⁺/CD83⁺ mature DCs that are unperturbed by the anti-CD83 Ab. These mature DCs can be immediately used as a source of DCs for DC-based vaccine strategies.

In addition to isolation and purification of immature DCs or mature DCs following Flt3-L/GM-CSF administration, the DC-modulation factor regimen can be modified to skew for development of immunosuppressive granulocytes (CD11b⁺/Ly-6G⁺ myeloid suppressor cells). These suppressor cells can also be procured using a similar isolation technique as outlined above.

Freezing and thawing has negligible effect on viability or function of DCs (Westermann J et al. Cancer Immunol Immunother, 52: 194-198, 2003). The purified mature DCs are cryopreserved and recovered using an established protocol for murine DCs (Sai T et al. J Immunol Methods, 264: 153-162, 2002).

### Example 14

### Schema for DC-Modulation Factor(S) Administration

Examples 5-13 employed administration of Flt3-L (or FLex) and/or GM-CSF by HTV and HLV gene delivery of factor-expressing pDNAs. Where sequential delivery is indicated, a span of 10 days between Flt3-L and subsequent GM-CSF delivery was used. The amount of DC-modulation factor and any timing between deliveries of different DC-modulation factors may have a potential impact on the expansion and maturation of DCs in vivo. A method to evaluate and optimize the parameters important in a schema for administration of DC-modulating factors is presented below, in the context of a HTV gene delivery-based schema. This optimization procedure can also be applied to develop a schema involving gene delivery- or protein-based administration of DC-modulating factors.

A. Flt3-L dose-response curve. Groups of C57BL/6 mice (n=5) receive 5, 10, 20, 40, or 80 μg or more Flt3-L pDNA by HTV delivery on d0 followed by sacrifice and spleen harvest on d10. Group-pooled splenocytes are analyzed by flow cytometry for the frequency and absolute number of CD11c⁺ DCs, as described for Table 1. Increasingly higher doses of Flt3-L pDNA are tested until the maximal expansion in splenic CD11c⁺ DCs is determined.

B. Flt3-L time-course study. Groups of mice receive the minimum pDNA dose of Flt3-L pDNA that promotes maximum expansion of splenic CD11c⁺ cells as determined in A. The frequency and absolute number of splenic CD11c⁺ cells is analyzed by flow cytometry every other day beginning on d4 through d20.

C. GM-CSF dose-response curve. Unlike Flt3-L gene delivery, GM-CSF promotes the appearance of a substantial number of splenic CD83⁺/CD86⁺ mature DCs. A dose-response study determines the lowest dose of mGM-CSF pDNA that results in the maximum number of mature DCs on d10 following HTV gene delivery. The dose-response analysis is performed in a similar fashion as described for Flt3-L (A, above) using 2, 4, 8, 16, 32 or more μg of mGM-CSF pDNA.

D. Sequential delivery interval. The time of mGM-CSF delivery relative to the time of maximal mFlt3-L-induced CD11c⁺ expansion is varied to determine the greatest expansion of splenic CD11c⁺/CD83⁺/CD86⁺ mature DCs. Groups of mice receive optimal mFlt3-L pDNA dose on d0 (determined in A), followed by the optimal mGM-CSF pDNA dose (determined in on days −4, −2, 0, +2 and +4 of the day of maximal Flt3-L-induced CD11c⁺ expansion (determined in B). Splenocytes are collected 4 days after mGM-CSF delivery and analyzed by flow cytometry for the frequency and absolute number of CD11c⁺/CD83⁺/CD86⁺ mature DCs.

E. mature DC induction time course. The time interval between GM-CSF delivery and splenocyte collection can influence the number of splenic DCs that can be harvested. Once expansion of mature DCs has peaked, normal homeostasis will be eventually re-established. Following delivery of mFlt3-L and mGM-CSF, spleens are collected on day 4, 8, 12, 16, and 20 post-GM-CSF gene delivery. Group-pooled splenocytes are analyzed by flow cytometry for the frequency and absolute number of CD11c⁻/CD83⁺/CD86⁺ mature DCs.

F. Effect of mCD40-L. Since CD40-triggering on DCs can be a critical late-stage event in DC maturation (Schuurhuis D H et al. Int Arch Allergy Immunol. 140:53-72 2006), the inclusion of CD40-L in the treatment schema may be aid in maximizing mature DC expansion or the ability to influence the immune response in a desired manner (such as optimal induction of antitumor immunity with a combined DC-modulation schema plus tumor vaccine strategy). CD40-L pDNA (such as pSP-D-CD154) is delivered concurrently with mGM-CSF pDNA, as both factors predominantly promote the maturation of immature DCs. Using the optimized parameters outlined in A-E, groups of mice are treated with the optimal mature DC-expansion protocol as developed in the previous steps. In addition to the optimal mGM-CSF pDNA dose, mice receive 1, 3, 10, 30 or 100 μg mCD40-L pDNA. The level of splenic CD11c⁺/CD83⁺/CD86⁺ mature DCs is determined by flow cytometry. CD40-L effects on vaccine-induced immunity is evaluated by combining the DC-modulation schema (with and without inclusion of CD40-L) with a hgp100 vaccination and determining the relative impact on generation of hgp100₂₅₋₃₃/H2D^{b}-tetramer⁺CD8⁺ T cells in the peripheral blood.

### Example 15

### Functional Characterization of in Vivo Expanded CD11c⁺/CD83⁺ Mature DCs

To be considered as a viable tool for DC-based research, the CD83⁺ mature DCs must be capable of effective Ag presentation to naïve CD4⁺ and CD8⁺ T cells. DC-T cell interaction is assessed in vitro by stimulation of Ag/peptide-specific T cells from transgenic mice. Specifically, the ability of pre- and post-cryopreserved CD83⁺ mature DCs to activate CD4⁺ and CD8⁺ OVA-specific naïve T cells from T cell receptor (TCR)-transgenic mice is performed.

Stimulation of Balb/c CD4⁺ T cells from DO11.10 mice (TCR specific for the I-A^{d}-restricted OVA₃₂₃₋₃₃₉ peptide). To demonstrate that CD83⁺/CD86⁺ mature DCs are able to activate naïve CD4⁺ T cells, T cells from the well-characterized DO11.10 TCR-transgenic mouse model, where all CD4⁺ T cells from these mice express a T cell receptor (TCR) specific for the OVA₃₂₃₋₃₃₉ peptide (Hopken U E et al. Eur J. Immunol. 35:2851-63 2005) are used. This system provides a powerful tool to study Ag-specific DC-T cell communication, as DO11.10 CD4⁺ T cells proliferate vigorously and produce cytokines (IFNγ, IL2, and IL4) in response to the combination of DC plus Ag. Co-culture of Balb/c (H2^{d} background) CD83⁺ mature DCs and DO11.10 CD4⁺ T cells in the presence of OVA₃₂₃₋₃₃₉ peptide Ag is used to assess T cell activation by in vitro cellular proliferation assay and IFNγ production.

CD4⁺ T cells are obtained from spleens of DO11.10 transgenic mice by 2 sequential magnetic bead separations, followed by depletion of 1-A⁺ contaminating APCs by anti-MHC class II (I-A) microbeads. The resulting splenic T cell preparations contain >95% CD4⁺ cells and used without further enrichment. Purity is monitored by flow cytometry for cells double-staining with anti-CD4 and mAb KJ1-26 that recognizes the transgenic TCR complex specific for the OVA₃₂₃₋₃₃₉ peptide. CD83⁺ mature DCs are generated in Balb/c mice using the optimal sequential Flt3-L/GM-CSF/CD40-L HTV gene delivery schema identified for C57BL/6 mice in Example 14, isolated and cryopreserved using the CD83-enrichment process described in Example 13. For assessing cell proliferation as a measure of T cell activation, freshly isolated DO11.1 CD4⁺ T cells (2.5×10⁵ cells/ml) are co-cultured in complete RPMI 1640 media with thawed Balb/c CD83⁺ mature DCs (5×10⁴ cells/ml) and 2-5 μg/ml OVA peptide (Ag specific or control peptide). Following 3 days of co-culture, cell proliferation is determined using the non-radioactive CellTiter-Glo Luminescent Cell Viability assay. As an additional correlate to evaluate CD4⁺ T cell activation in this system, we determine IFNγ production by DO11.10 T cells, measured by ELISA (R & D Systems) of supernatants from parallel co-cultures. As demonstrated by others (Matsue H et al. J Immunol, 169: 3555-3564, 2002), IFNγ production and T cell proliferation is detected in co-cultures which posses 3 components: functional DCs, DO11.10 CD4⁺ T cells, and OVA₃₂₃₋₃₃₉ peptide. Unfractionated DO11.10 splenocytes+peptide are included as positive controls. Table III describes the combination of cells and peptide Ags that will be tested and the expected cell proliferation and IFNγ production results.

Activation of C57BL/6 CD8⁺ T cells from OT-1 mice (TCR specific for the H-2^{b}-restricted OVA₂₅₇₋₂₆₄ peptide). In a similar fashion, we show that CD83⁺ mature DCs are capable of stimulating CD8⁺ T cells using T cells from OT-1 TCR transgenic mice. Utilizing OT-1 CD8⁺ T cells in a co-culture system with DCs plus Ag peptide provides a convenient method for assessing Ag-specific CD8⁺ T cell activation that is easily measured by supernatant IFNγ levels (Strome S E et al. Cancer Res, 62: 1884-1889, 2002). OT-1 TCR transgenic mice (Vα2/Vβ5.1) express the TCR specific for the H-2^{b}-restricted OVA₂₅₇₋₂₆₄ peptide on all CD8⁺ T cells. To obtain Ag-specific CD8⁺ T cells, spleen and lymph nodes (axillary and inguinal) from OT-1 TCR transgenic mice are harvested, homogenized, pooled, and passed over a CD8-negative selection column, followed by depletion of I-A⁺ contaminating APCs by anti-MHC class II (I-A) microbeads. Purity is monitored by flow cytometry using a labeled tetramer specific for the H-2^{b}-restricted OVA₂₅₇₋₂₆₄ peptide TCR (Nugent C T et al. Immunol Lett, 98: 208-215, 2005). Using the optimal mature DC-expansion schema identified for C57BL/6 mice in Example 14, enriched CD83⁺ mature DCs are used for CD8⁺ T activation as fresh and post-cryopreserved mature DCs. For co-culture with OT-1 CD8⁺ T cells, the OVA₂₅₇₋₂₆₄ peptide is the activating Ag, while the OVA₃₂₃₋₃₃₉ peptide serves as the control peptide. This series of co-cultures is performed in an identical manner as outlined above for the DO11.10 CD4⁺ T cells with only IFNγ production being the indicator of CD8⁺ T cell activation. Supernatants are evaluated for IFNγ production after 24 and 48 hrs.

### Example 16

### Modulation of Regulatory T Cells

The maturation state of DCs may be important in the peripheral development of regulatory T cells (Tregs), with predominantly immature DCs promoting their activation and expansion. Cancer vaccine strategies that expose the immune system to TAA in the context of predominantly immature DCs may skew the ensuing immune response toward development of Tregs that suppress execution of clinically meaningful antitumor responsiveness. The combinatorial Flt3-L/GM-CSF plus hgp100 HLV vaccine scheme delivers the gp100 TAA to a tissue compartment with a dramatic influx of mature DCs (Example 9). Thus, an important consideration is whether this HLV vaccine scheme has additional antitumor benefit by modulating subsequent Tregs development. As tumors progress in mice, the percentage of Tregs increases dramatically inside the tumor, although it remains constant in the spleen and draining lymph nodes. Intratumoral depletion of Tregs can unmask tumor immunogenicity and lead to resolution of late-stage disease. Therefore, can analyze tumor-infiltrating lymphocytes (TILs) for changes in the percentage of intratumoral Treg cells that can occur as a result of the DC-expansion aspect of a combinatorial (i.e., combination of optimal HLV gene delivery DC-expansion as determined by studies similar to those described in Example 14, plus HLV vaccination) hgp100 HLV vaccination procedure. To demonstrate that the combinatorial HLV hgp100 vaccine protocol reduces Treg development, the number of intratumoral Tregs in the murine B16 melanoma tumor TIL population are compared between no treatment control, HTV Flt3-L+hgp100 vaccinated (HTV delivery of mFlt3-L only results in predominantly immature DC expansion and will promote Treg development), and optimal HLV mature DC expansion+hgp100 vaccinated mice. Following TIL enrichment from excised subcutaneous B16 tumors, the number of Tregs is assessed by 3-color flow cytometry to identify CD4⁺ CD25⁺FoxP3⁺ regulatory T cells.

To ensure B16 tumor development in all control and vaccinated mice, animals are injected with a high dose (5×10⁶) of B16 tumor cells on d10 (with d0 being the initial treatment date when Flt3-L pDNA is delivered). B16 tumors are harvested on d2, 4, and 8 following hgp100 pDNA delivery in HLV vaccinated mice. For the isolation of T cells from the B16 tumor tissue, mice are initially bled to decrease the contamination of tumor tissue by blood. Tumor tissues is collected, cut into pieces and resuspended in Dulbecco's modified Eagle's medium supplemented with 2% fetal calf serum and 1.5 mg/ml of collagenase D for 20-60 min in a 37° C. shaking incubator until all of the tumor tissue is resolved into a single-cell suspension. Group-pooled TIL cells are enriched from this single-cell suspension with biotin-conjugated Thy-1.2 antibody followed by antibiotin magnetic beads using the MACS system (Miltenyi Biotech). Enriched tumor-infiltrating T cells are fixed in 1% paraformaldehyde and 0.05% Tween-20 overnight at 4° C. and treated twice with DNAse. Cells are incubated with anti-mouse-FoxP3-PE (clone FJK-16s: eBioScience) for 1 h for detection of intracellular FoxP3 protein. Dual cell surface staining for murine CD4 and CD25 is done using anti-mouse-CD4-APC (BD Bioscience) and anti-murine-CD25-FITC (BD Bioscience). Stained cells are analyzed by flow cytometry and the numbers of CD4⁺CD25⁺FoxP3⁺ cells (i.e., Treg cells) per gram of tumor tissue were determined. The combinatorial HLV vaccine protocol is considered effective in reducing Treg development as there is a statistically significant (p<0.01) reduction in the number of intratumoral CD4⁺CD25⁺FoxP3⁺ cells as compared to controls.

### Example 17

### Immunocytokine Therapy

Therapeutic monoclonal antibodies (mAbs) will preferentially localize and bind to tumor cells. Upon binding, the Fc fragment of the mAb can activate two mechanisms of tumor cell destruction: 1) through activation of the complement cascade and 2) via the process of antibody dependent cellular cytotoxicity (ADCC) by interacting with Fc receptor+immune effector cells, such as NK cells. Interleukin 2 (IL2) administration has been a promising adjunct to enhance ADCC. Linking IL2 directly to mAbs results in increased levels of IL2 concentrated at the tumor microenvironment with a subsequent decrease in toxicity related to systemic IL2 therapy. The resulting fusion molecules, known as immunocytokines (ICs), have shown far more potent anti-tumor effects than the same amounts of mAb and IL2 given as separate molecules (Lode H N et al. 1997, Sondel P M et al. 2003). Clinical and preclinical evaluation indicated that ICs predominantly mediate tumor destruction through NK-dependent mechanisms (King D M, et al. 2004, Neal Z C et al. 2004). Tumor cell susceptibility is directly correlated by the MHC molecule expression level on the target cell (Imboden M et al. 2001). IC-mediated antitumor responses rarely induce durable antitumor memory. ICs include other, additional engineered therapeutic antibody/cytokine fusion molecules.

The hu14.18-IL2 IC is comprised of the humanized 14.18 mAb which recognizes the GD₂ disialoganglioside expressed on certain neuroectodermally derived tumors, including NB and melanoma (Reisfeld R A 1992), and a human IL2 molecule linked to the carboxy-terminus of each human IgG1 heavy chain (Gillies S D et al. 1992). In A/J mice bearing the NXS2 murine neuroblastoma, administration of the chimeric IC (ch14.18-IL2; an earlier predecessor of hu14.18-IL2) has a greater anti-NXS2 effect than treatment with IL2 alone, the ch14.18 mAb alone, or combined therapy with the ch14.18 mAb together with IL2 (Lode H N et al. 1997, (Lode H N et al. 1998). These antitumor effects of ch14.18-IL2 against NXS2 NB are completely dependent upon NK cells and do not require T cell involvement (Lode H N et al. 1998). IC treatment provided early after tumor establishment, at a sufficient dose, can promote complete resolution of all detectable tumor, with long term survival for at least some animals. If IC treatment is provided at a lower dose or after the tumors have had a longer time to establish, many animals show tumor shrinkage, only to be followed by a delayed recurrence or outgrowth of progressive tumor (Neal Z C et al. 2004).

The KS-IL2 IC targets the human epithelial cell adhesion molecule (EpCAM), which is overexpressed on most epithelial carcinomas, including colon, lung, prostate, ovarian, and breast cancers. A recent study found that primary and metastatic breast cancers express EpCAM at levels 100 to 1000-fold higher than normal breast tissue (Osta W A et al. 2004). Accordingly, breast cancer should be effectively targeted by KS-IL2 treatment. Phase I clinical trials of KS-IL2 for treatment of ovarian, colon, and prostate cancer are currently recruiting patients. It should be noted, however, that KS-IL2 therapy against any EpCAM⁺ cancer would be constrained in antitumor efficacy by the dose-limiting IL2-related toxicities (King D M, et al. 2004, Osenga K L et al. 2006). Consequently, any adjuvant treatment that could enhance the anti-tumor effects of IC therapy would be of great clinical benefit, as has been demonstrated preclinically with the addition of chemotherapy (Holden S A et al. 2001), systemic IL2 (Neal Z C et al. 2004), IL12 gene therapy (Lode H N et al. 1998), and antiangiogenic compounds (Lode H N et al. 1999). Here, we propose to significantly increase the number of NK effector cells prior to KS-IL2 therapy.

### Example 18

### Flt3-L Plus IC Treatment

Flt3-L is a hematopoietic stem cell growth and differentiation factor that acts on CD34⁺ progenitor cells and induces in vivo expansion of dendritic cells (DC) and NK cells when administered as protein (Shaw S G et al. 1998) or by gene therapy (He Y et al. 2000). Flt3-L treatment may induce an NK- or T cell-dependent antitumor response; the latter may result in durable antitumor memory (Silver D F et al. 2000). For example, breast cancer patients exhibited a higher frequency of interferon γ-secreting HER-2/neu-specific T cells when peptide vaccinated in combination with Flt3-L treatment (Disis M L et al. 2002).

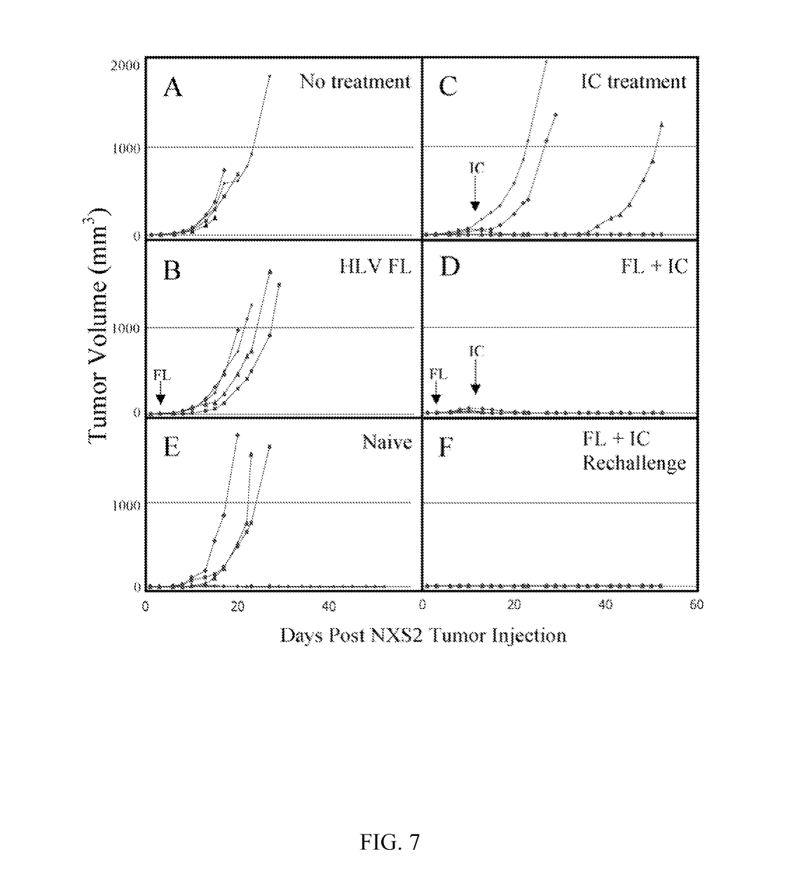

In studies with the NXS2 murine neuroblastoma model, treatment with hu14.18-IL2 IC (targeting the GD2 disialoganglioside on NXS2) or Flt3-L were each effective at resolving established tumor in mice, but often failed to prevent tumor recurrence resulting from TEV (Neal Z C et al. 2004). We have demonstrated that combining IC plus Flt3-L had greater antitumor benefit than either single agent treatment alone (Neal Z C et al. Cancer Immunol Immunother 2007). Flt3-L was expressed in vivo using the clinically applicable (Wells D J 2004) non-viral intravenous hydrodynamic limb vein (HLV) gene delivery procedure, a technique developed by Mirus Bio (Hagstrom J E et al. 2004). Our results indicate that Flt3-L (FIG. 7B) or hu14.18-IL2 (FIG. 7C) treatment alone resulted in delayed NXS2 tumor progression as compared to the no-treatment control group (FIG. 7A).

Mice that received Flt3-L plus IC exhibited the greatest antitumor benefit with all mice showing complete and sustained resolution of their measurably established NXS2 tumors (FIG. 7D). Supplying Flt3-L by HLV gene delivery resulted in a ˜4-fold increase in the number of DX5⁺ splenic NK cells (see Table 3, Example 12), suggesting that enhanced resolution of the primary tumor with the combinatorial regimen is, in part, the result of a greater number of NK effectors available to facilitate IC-mediated ADCC. Furthermore, mice treated with the combined Flt3-L plus IC regimen exhibited durable antitumor memory and were completely protected when rechallenged with NXS2 tumor 70 days later (FIG. 7E). Since Flt3-L protein treatment of NXS2 tumors is able to induce T cell-dependent antitumor memory (Neal Z C et al. 2004), the protective memory response is likely T cell-dependent as well. Flt3-L gene therapy in these studies also promoted a significant increase in CD11c⁺ splenic DCs (see Table 3, Example 12). These cells should be capable of presenting numerous tumor antigens taken up from IC-killed tumor cells. The antitumor memory response may therefore involve a tripartite interaction between the resolving tumor mass and the Flt3-L-expanded pool of NK and DC cells (Disis M L et al. 2002).

### Example 19

### Combined Flt3-L Gene Therapy Plus Immunocytokine Treatment Results in Enhanced Expansion of NK and DC Cell Populations

While Flt3-L gene therapy administered by HLV delivery clearly promoted the expansion of NK and DC cells (Table 3, Example 12), greater antitumor activity was achieved when Flt3-L HLV gene therapy was combined with IC treatment (Example 18).

TABLE 4
Combined Treatment

| | Cells/ | | | | | | |
|---|---|---|---|---|---|---|---|
| | spleen^{b} | Splenic phenotypic profile^{c}: | | | | Total DCs^{d} | Total NKs^{e} |
| Treatment^{a} | ×10⁶ | % CD4⁺ | % CD8⁺ | % CD11c⁺ | % DX5⁺ | ×10⁶ | ×10⁶ |
| 1. No Treatment | 38 | 14.5 | 6.3 | 1.8 | 4.5 | 0.7 | 1.7 |
| 2. hu14.18-IL-2 | 113 | 12.6 | 5.8 | 2.6 | 9.6 | 2.9 | 10.8 |
| 3. mFL (Both) | 79 | 13.6 | 7.1 | 3.2 | 5.9 | 2.5 | 4.6 |
| 4. mFL + hu14.19-IL-2 | 149 | 14.8 | 8.4 | 4.3 | 10.8 | 6.4 | 16.1 |
| 5. rhIL-2 | 130 | 13.3 | 9.3 | 1.8 | 12.8 | 2.3 | 16.6 |

^{a}Groups (n = 4) of A/J strain mice received treatment as follows: (1) no treatment, (2) 10 μg/d of hu14.18-IL-2 IC days 7-10, (3) HLV delivery of 200 μg mFLex DNA to right limb plus 200 μg mFL DNA to left limb on d 0, (4) combination of treatments described in (2) and (3), and (5) 140,000 I.U./d of rhIL-2 by constant infusion osmotic pump on days 8-11.
^{b}Spleens were harvested on d 11 and pooled for each group. Following erythrocyte lysis by hypotonic shock, the number of viable cells was determined. Values are ×10⁶ cells/spleen.
^{c}Isolated splenocytes were pooled for each group and stained with primary-conjugated mAbs (BD Biosciences, San Diego, CA) to murine CD4, CD8, CD11c, and DX5. Value represents the percent of viable splenocytes positive for specific cell-surface staining.
^{d}Indicates the total number of CD11c⁺ DCs per spleen and is determined by the formula: number of viable cells/spleen × % CD11c⁺ cells. Values are ×10⁶ cells/spleen.
^{e}Indicates the total number of DX5⁺ NKs per spleen and is determined by the formula: number of viable cells/spleen × % DX5⁺ cells. Values are ×10⁶ cells/spleen.

Mice that received the combinatorial treatment (group 4, Table 4) exhibited the highest degree of splenomegaly, with a near 4-fold increase in the number of splenocytes (149×10⁶ cells/spleen) as compared to no-treatment controls (38×10⁶ cells/spleen). The combinatorial treatment induced NK expansion (16.1×10⁶ cells/spleen) that was greater than IC or mFL alone, and comparable to that induced by 4 d of constant infusion IL-2 (group 5). Furthermore, the combinatorial treatment induced the highest observed increases in the frequency of splenic DCs, reaching 4.3%. Thus, the biological effect of combining FL gene therapy with IC treatment induced an even greater expansion in the absolute number of DC (6.4×10⁶ cells/spleen) while promoting the maximally observed expansion of NK cells. Thus, the combinatorial FL plus IC treatment regimen enabled a potent and durable antitumor response against an established primary tumor burden and effectively vaccinated animals from subsequent cognate tumor challenge (FIG. 1, Example 18A).

### Example 20

### FL Gene Therapy Plus IC Treatment Reduces Development of Tumor-Escape Variants (TEVs)

In an earlier study with NXS2 tumors (Neal Z C et al. 2004), treatment with FL protein induced a T cell-dependent antitumor response which promoted the development of MHC class I H2-depressed TEVs. Conversely, IC treatment mediated an NK-dependent antitumor response, which resulted in H2-elevated TEVs.

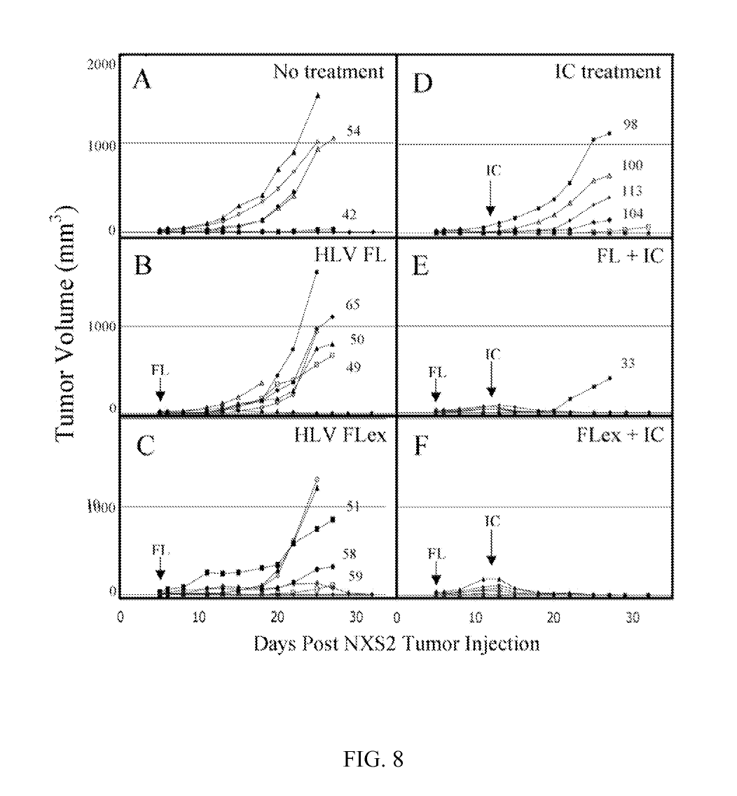

At day 27 following tumor engraftment, detectable NXS2 tumors were collected and assessed for MHC class I H2D^{d} expression by flow cytometry. The specific Mean Fluorescent Intensity (sMFI) ratio for the H2D^{d} expression level on each individually excised NXS2 tumor is represented as a numerical value adjacent to the graph showing the growth of that specific harvested tumor in FIG. 8. The sMFI ratio for H2D^{d} expression on cultured NXS2 cells was 15. Tumors from untreated mice exhibited a relative increase in H2D^{d} expression (FIG. 8A: sMFI ratio of 54 and 42 for two individually excised NXS2 tumors; mean sMFI ratio value of 48) compared to cultured NXS2 cells. The sMFI H2D^{d} ratios for tumors from the mFL gene therapy treatment groups were similar to the sMFI H2D^{b} ratios for tumors from the control group (FIG. 8B: sMFI ratios=49, 50, and 65; mean=55) or mFLex (FIG. 2C: sMFI ratios=51, 58, and 59; mean=56). In contrast, the ratios for tumors from the IC treatment group were notably elevated (FIG. 8D: sMFI ratios=98, 100, 104 and 113; mean=104). There was a significant statistical difference (p=3×10⁻⁶) in sMFI H2D^{d} ratio values from tumors obtained from the mFL and mFLex gene therapy treated mice when compared to those obtained from the IC treated animals. The single recurrent tumor that developed from an animal in the mFL gene therapy plus IC treatment group exhibited the lowest sMFI ratio (33) from any of the recovered tumors. Analysis of MHC class I expression on tumors that progressed following immunotherapy indicates that the effects of IC-induced immunoediting may be occurring during or shortly following IC treatment. Thus, tumors harvested 12 days after completion of IC treatment were already exhibiting evidence of immunoediting as their group mean H2D^{d} sMFI value was 104.

Recent evidence has clearly demonstrated that the close interface between DCs and NK cells may be instrumental in fully activating NK-dependent innate immune responses, as well as inducing DC maturation events critical in orchestrating the development of adaptive T cell-dependent immunity (Munz C, Steinman R M, Fujii S. J Exp Med 202:203-207, 2005). Flt3-L administration resulted in a dramatic increase in DC and NK cells (Example 12), which was even more pronounced when followed by IC treatment. IC is expected to interact with the expanded pool of NK cells to mediate a greater early antitumor response against established primary tumor, due in part, to antibody dependent cellular cytotoxicity (ADCC) by the increased number of NK effectors available. The IL-2 component of the IC molecule activates these NK effectors to mediate ADCC and to produce the proinflammatory factor, IFNγ This IFNγ in the tumor micro-environment increases MHC class I expression on any viable tumor targets still remaining and is expected to cause them to become less susceptible to NK-dependent antitumor effects. The increased MHC class I expressed by tumor will then boost tumor immunogenicity if DCs are present. Additionally, the NK-derived IFNγ should promote maturation of the expanded pool of DCs and contribute to NK-DC cross-talk. The maturing DCs would be expected to produce IL-12, further activating the IFNγ-producing NK effectors involved in the early antitumor response. It is known that NK-mediated tumor rejection can induce tumor-specific T cell memory. Thus, the destruction of the primary tumor is mostly accomplished by NK effectors and provides a reservoir of antigenic tumor material for accumulation and presentation by the maturing pool of DCs. This should elicit a more effective adaptive tumor-specific CTL and T cell memory response, which is able to break tolerance for tumor-expressed self-antigens. Those tumor cells which escaped the ensuing NK-dependent response by up-regulating their MHC class I expression should also become more immunogenic and susceptible to recognition and elimination by the adaptive T cell response.

### Example 21

### Cancer Treatment

Treatment of tumors with IC or DNA vaccination is effective at recruiting participation of innate and adaptive antitumor immunity, respectively. As individual treatment modalities, each strategy activates critically important, although distinctively complementary, mechanisms of tumor destruction that may each select for distinct forms of TEV through immunoediting (Neal Z C et al. 2004). Combining these modalities provides greater antitumor efficacy and diminished TEV development. Above was shown that IC-mediated tumor resolution and DNA cancer vaccine efficacy can both be dramatically improved by delivery of Flt3-L. Thus IC plus DNA vaccination treatment, preferably with UTAs, and enhancement with Flt3-L treatment provides a more effective antitumor strategy.

IC plus DNA vaccine treatment induces an antitumor response involving simultaneous NK- and T cell-dependent effector mechanisms against cancer. These mechanistically distinct and independent activities together enhance resolution of existing tumor burden, while diminishing TEV development. This effect is enhanced by Flt3-L gene therapy through in vivo expansion of NK and DCs. The larger pool of NK effectors enables greater IC-mediated cancer cell killing. This immediate IC-directed tumor resolution provides tumor antigen for presentation by the Flt3-L-expanded population of DCs which subsequently activate tumor-specific T cell responses necessary for durable immunity. The pool of Flt3-L-expanded DCs is matured as a consequence of the additional GM-CSF gene therapy. These mDCs enhance DNA vaccine efficacy and result in a higher frequency of TERT-specific antitumor CTL effectors to mediate enhanced tumor destruction and durable immunity.

KS-IL2 IC treatment targets EpCAM-expressing cancer cells and mediates an NK-dependent antitumor response. Flt3-L gene therapy expands the number of NK cells and enhances KS-IL2-mediated antitumor effects, as well as promotes the development of cancer-specific T cell-dependent antitumor memory (Bubenik J 2003). Tolerance against TAA can be broken by xenogeneic genetic vaccination to enable TAA-specific CTL activity. Vaccine efficacy is enhanced by employing a mDC expansion procedure involving Flt3-L/GM-CSF gene therapy (Smyth M J et al. 2002). The combinatorial approach involving Flt3-L/KS-IL2 plus xenogeneic vaccination results in enhanced NK-mediated and T cell-mediated antitumor impact against primary tumor, and reduces cancer TEV development and inhibits recurrent disease.

A. Flt3-L gene therapy augments KS-IL2 IC treatment against established 4T1-EpCAM tumors and enables development of protective T cell-dependent antitumor memory. Flt3-L therapy can enhance the immediate KS-IL2-mediated antitumor response against 4T1-EpCAM tumors by expanding the pool of NK effectors. This combined regimen also facilitates development of a protective T cell-dependent antitumor memory response against 4T1-EpCAM and 4T1 tumor challenge through antigen presentation by Flt3-L-expanded DCs. These DCs use KS-IL2-killed tumor as the source of tumor antigen to prime tumor-specific naïve T cells.

B. Xenogeneic hTERT DNA vaccine against 4T1 tumors. Genetic vaccination by hTERT DNA provides prophylactic and therapeutic T cell-dependent immunity against 4T1-EpCAM and 4T1 breast cancer. Vaccination is done concurrently with in vivo expansion of mDCs by sequential Flt3-L plus GM-CSF adjuvant gene therapy.

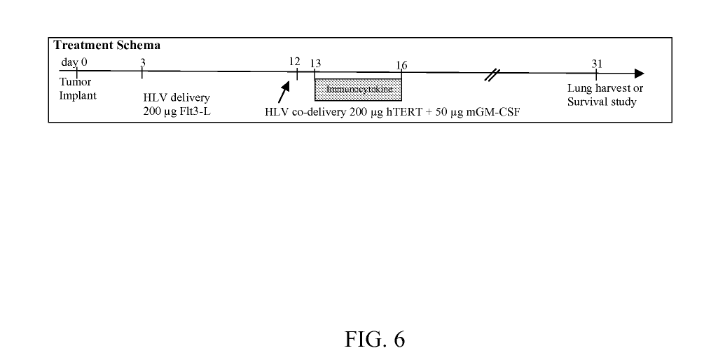

C. Combinatorial Flt3-L/KS-IL2 plus hTERT vaccination for induction of antitumor benefit. Combinatorial KS-IL2 IC/Flt3-L plus the TAA/Flt3-L/GM-CSF DNA vaccine strategy against established 4T1-EpCAM tumors provide immediate and long-term antitumor benefit. This combination minimizes TEVs. The described combinatorial KS-IL2/Flt3-L therapy plus hTERT/Flt3-L/GM-CSF DNA vaccination strategy can be performed using two gene delivery procedures. The first gene delivery (about day 3) is for murine Flt3-L pDNA (for the disease model); the second (about day 12) for co-delivery of murine GM-CSF plus TAA pDNA. The single HLV delivery of Flt3-L is mutually utilized by KS-IL2 and TAA vaccine therapies. The basic treatment schema is depicted in FIG. 6, with actual time points depending on disease and subject.

D. Flt3-L gene therapy augments IC treatment against established tumors and enables development of protective T cell-dependent antitumor memory.

Maximal NK expansion and activity following HLV Flt3-L pDNA delivery. Animal receive an appropriate does of Flt3-L expression vector on d 0. Maximal NK expansion and activity is then measured by standard methods in the art, such as by harvesting PBMCs and spleens on various days (for example, d 8, 10, 12, or 14) and analyzing cell populations using flow cytometry and NK or ADCC killing assays.

Immunocytokine treatment (such as KS-IL2 treatment) is initiated at maximal NK activity. The precise pDNA dose and administration time are determined for the type of cancer and host animal. Preferably, the dose of IC that mediates the greatest antitumor impact is used. Studies with showed 4T1-EpCAM in mice showed that 15-30 μg/d of KS-IL2 was not curative against these tumors and that IC doses as high as 300 μg were well tolerated (Holden S A et al. 2001). IC can be administered at multiple times starting just before, at, or just after maximal Flt3-L induced maximal NK activity. IC/Flt3-L therapy may induce an NK-dependent immediate antitumor response and a T cell-dependent memory response.

E: Xenogeneic UTA DNA vaccine against cancer. UTA/Flt3-L/GM-CSF DNA vaccine can be used as a prophylactic against tumor recurrence. Flt3-L in administered on day 0. At maximal DC expansion or NK activity (about day 10), UTA (such as TERT)+mGM-CSF are administered to the host. UTA and mGM-CSF can be administered again on about day 20 to provide a vaccine boost (as is typical in the art)

F. Combinatorial IC/Flt3-L plus UTA/Flt3-L/GM-CSF vaccination for induction of antitumor benefit. Combination of the independent IC/Flt3-L and UTA/Flt3-L/GM-CSF immunotherapies into a single comprehensive approach is used to enhance overall efficacy. The separate IC/Flt3-L and UTA/Flt3-L/GM-CSF therapies are unified by an initial Flt3-L administration. IC (such as KS-IL2), UTA (such as TERT), and GM-CSF are administered at the appropriate times points after Flt3-L administration as described above (about 10 days for UTA and GM-CSF). IC/Flt3-L therapy is able to induce T cell-dependent antitumor memory. This therapy is expected to elicit tumor-specific T cell responses against undefined TAAs distinct from the UTA-specific T cell response triggered by the UTA/Flt3-L/GM-CSF treatment. Flt3-L therapy provides a greatly expanded pool of NK cells for IC therapy. Flt3-L plus GM-CSF gene therapy greatly expands the number of mDCs available for cancer vaccines. Use of the UTAs (universal tumor antigens) limits TEV (tumor escape variant) development. Genetic vaccination following Flt3-L plus GM-CSF gene therapy provides long lasting immunity. The combination of Flt3-L gene therapy, IC therapy, and GM-CSF+UTA genetic vaccination provides an combinatorial treatment with improved in tumor destruction and immune memory. The IC, Flt3-L, UTA, and GM-CSF proteins can be administered by injection of purified proteins are through delivery of gene encoding these proteins.

The leading cause of death in many cancer patients is metastatic burden. This progression in disease status shows heavy reliance on immunoediting and TEV development. The combination of two distinct and complementary approaches (NK-mediated IC therapy and CTL-mediated cancer vaccines), each enhanced by Flt3-L therapy, provides improved antitumor benefit and TEV avoidance.

We claim:

1. A method for enhancing an immune response to a tumor in an animal comprising:
a) administering an effective dose of Fms-like tyrosine kinase ligand (Flt3-L) to the subject;
b) providing a time interval during which a population of immature dendritic cells is expanded in the subject;
c) administering, after the time interval of step b), an effective does of granulocyte-monocyte colony stimulating factor (GM-CSF),
d) administering an effective does of an immunocytokine to the subject; and,
e) administering an effective does of a tumor associated antigen to the subject.

2. The method of step 1 wherein the time interval consists of about 5 to about 15 days.

3. The method of step 1 wherein the time interval consists of about 8 to about 12 days.

4. The method of step 1 wherein administering an effective dose of Flt3-L consists of delivering to the subject purified Flt3-L protein.

5. The method of step 1 wherein administering an effective dose of Flt3-L consists of delivering to the subject a nucleic acid encoding the Flt3-L.

6. The method of step 1 wherein administering an effective dose of GM-CSF consists of delivering to the subject purified GM-CSF protein.

7. The method of step 1 wherein administering an effective dose of GM-CSF consists of delivering to the subject a nucleic acid encoding the GM-CSF.

8. The method of step 1 wherein administering an effective dose of immunocytokine consists of delivering to the subject purified immunocytokine protein.

9. The method of step 1 wherein administering an effective dose of immunocytokine consists of delivering to the subject a nucleic acid encoding the immunocytokine.

10. The method of step 1 wherein administering an effective dose of tumor associated antigen consists of delivering to the subject purified tumor associated antigen protein.

11. The method of step 1 wherein administering an effective dose of tumor associated antigen consists of delivering to the subject a nucleic acid encoding the tumor associated antigen.

12. The method of claim 1 wherein the tumor associated antigen consists of a universal tumor associated antigen.

13. The method of claim 1 wherein the immunocytokine comprises of IL12.
